(19) United States
(12) Patent Application Publication
(10) Pub. No.: US 2020/0369767 A1
(43) Pub. Date: Nov. 26, 2020

(54) REGIMES FOR CO-ADMINISTRATION OF IMMUNOTHERAPEUTIC AGENTS AGAINST C-KIT AND CD47

(71) Applicant: Forty Seven, Inc., Foster City, CA (US)

(72) Inventors: Craig Gibbs, Palo Alto, CA (US); Jens-Peter Volkmer, Foster City, CA (US); Irving L. Weissman, Stanford, CA (US); Kristopher Marjon, Foster City, CA (US)

(21) Appl. No.: 16/880,661

(22) Filed: May 21, 2020

Related U.S. Application Data
(60) Provisional application No. 62/852,901, filed on May 24, 2019.

(51) Int. Cl. C07K 16/28

(52) U.S. Cl. CPC C07K 16/2803; C07K 16/2896; A61K 45/06; C07K 2317/56; C07K 2317/24

(57) ABSTRACT

The invention provides co-administration regimes of immunotherapeutic agents specifically binding to c-kit or inhibiting CD47-SIRPα for ablation of endogenous HSPCs. Relatively low levels of anti-c-kit result in saturation of binding to c-kit on HSPCs without significant reduction of the levels HSPCs. Significant reduction of the level of HSPCs can be obtained when the action of anti-c-kit is promoted by an immunotherapeutic agent inhibiting CD47-SIRPα. HSPCs expressing c-kit can thus be reduced to an acceptable level an acceptable level to permit introduction of replacement HSPCs without detrimental delay during which a subject has inadequate HSPCs.

### CROSS-REFERENCE TO RELATED APPLICATION

The present application claims the benefit of U.S.62/852,901, filed May 24, 2019 incorporated by reference in its entirety for all purposes.

### SEQUENCE LISTING

The application includes sequences disclosed in txt file 547756SEQLST.txt of 38,645 bytes, named created May 21, 2002, which is incorporated by reference.

### BACKGROUND

Stem cells provide the means for organisms to maintain and repair certain tissues, through propagation to generate differentiated cells. Hematopoietic stem cell transplantation has been used to provide patients with the capacity to generate blood cells, usually where the patient has been ablated of endogenous hematopoietic stem cells by chemotherapy, or other conditioning regime.

Hematopoietic cell transplantation generally involves the intravenous infusion of autologous or allogeneic blood forming cells including hematopoietic stem cells. These are collected from bone marrow, peripheral blood, or umbilical cord blood and transplanted to reestablish hematopoietic function in patients whose bone marrow or immune system is damaged or defective. This procedure is often performed as part of therapy to eliminate a bone marrow infiltrative process, such as leukemia, or to correct congenital immunodeficiency disorders. Hematopoietic cell transplantation is also used to allow patients with cancer to receive higher doses of chemotherapy than bone marrow can usually tolerate; bone marrow function is then salvaged by replacing the marrow with previously harvested stem cells (see generally WO 2004/002425 and WO2018/140940).

### SUMMARY OF THE CLAIMED INVENTION

The invention provides methods of ablating hematopoietic stem and progenitor cells (HSPCs) in a patient in need thereof, comprising administering to the patient 0.15-2 mg/kg of an immunotherapeutic agent specifically binding to c-kit and an effective regime of an immunotherapeutic agent specifically binding to CD47 or SIRPα, wherein HSPCs are ablated in the patient.

Optionally, the patient is administered a single dose of 0.15-1 mg/kg of the immunotherapeutic agent specifically binding to c-kit. Optionally, the patient is administered the immunotherapeutic agent specifically binding to c-kit in multiple doses over a period of up to seven days delivering substantially the same area under the curve as a single dose of 0.15-1 mg/kg. Optionally, the patient is administered two doses of 0.15-1 mg/kg of the immunotherapeutic agent specifically binding to c-kit spaced by 3-7 days. Optionally, the effective regime of an immunotherapeutic agent specifically binding to CD47 comprises a first dose and a second dose higher than first dose. Optionally, the first dose is 1mg/kg and the second dose is 10-30 mg/kg, preferably 15-20 mg/kg. Optionally, the immunotherapeutic agent specifically binding to c-kit is administered as a single dose at the same time as the second dose of the immunotherapeutic agent specifically binding to CD47. Optionally, the single dose of the immunotherapeutic agent specifically binding to c-kit and the second dose of the immunotherapeutic agent specifically binding to CD47 are administered by co-infusion. Optionally, the second dose of immunotherapeutic specifically binding to CD47 and single dose of immunotherapeutic specifically binding to c-kit are administered 3-15 days, optionally 7 days after the first dose of the immunotherapeutic agent specifically binding to CD47. Optionally, the immunotherapeutic agent specifically binding to c-kit is administered in at least three doses over a period of 10-30 days. Optionally, the immunotherapeutic agent specifically binding to CD47 or SIRPα is administered on the same day as each dose of the immunotherapeutic agent specifically binding to c-kit is administered, optionally with an additional dose lower than and preceding the other dosages.

Optionally, the method further comprises introducing HSPCs into the patient. Optionally, HSPCs are introduced into the patient 5-15 days after the single dose of the immunotherapeutic agent specifically binding to c-kit and the second dose of the immunotherapeutic agent specifically binding to CD47 are administered.

Optionally, only the first and second doses of immunotherapeutic agent specifically binding to CD47 and the single dose of immunotherapeutic agent specifically binding to c-kit are administered before introducing the HSPCs. Optionally, the method further comprises administering a third dose of the immunotherapeutic agent specifically binding to CD47 after the second dose, optionally the second and third doses being the same amount of the immunotherapeutic agent. Optionally, multiple doses of the immunotherapeutic agent specifically binding to c-kit are administered and multiple doses of the immunotherapeutic agent specifically binding to CD47 or SIRPα are administered and the HSPC's are introduced into the patient 5-15 days after the last dose of the immunotherapeutic agent specifically binding to c-kit, or the immunotherapeutic agent specifically binding to CD47 or SIRPα, whichever is later. Optionally, the last dose of the immunotherapeutic agent specifically binding to c-kit and the last dose of the immunotherapeutic agent specifically binding to SIRPα are administered on the same day.

Optionally, the immunotherapeutic agent specifically binding to CD47 is an antibody specifically binding to CD47. Optionally, the immunotherapeutic agent specifically binding to CD47 is humanized 5F9, optionally magrolimab.

Optionally, an effective regime of an immunotherapeutic agent specifically binding to SIRPα is administered. Optionally, the immunotherapeutic agent specifically binding to SIRPα is an antibody. Optionally, the antibody comprises a heavy chain variable region having a sequence comprising SEQ ID NO:29 and a light chain variable region having a sequence comprising SEQ ID NO:30. Optionally, the antibody specifically binding to SIRPα is any of FSI-189, ES-004, BI765063, ADU1805, and CC-95251. Optionally, the antibody specifically binding to SIRPα is administered at a dose of 10-30 mg/kg. Optionally, a single dose of the antibody specifically biding to SIRPα is administered. Optionally, multiple doses of the antibody specifically binding to SIRPα are administered.

Optionally, the immunotherapeutic agent specifically binding to c-kit is an antibody. Optionally, the antibody is a humanized form of SR1 of human IgG1 isotype. Optionally, the antibody comprises a heavy chain variable region having a sequence comprising any of SEQ ID NOS:7-9 and a light chain variable region having a sequence comprising SEQ ID NO:10. Optionally, the heavy chain variable region has a sequence comprising SEQ ID NO:7.

Optionally, administration of the immunotherapeutic agents ablates c-kit positive HSPCs by 25-95% of the level before administration. Optionally, administration of the immunotherapeutic agents ablates c-kit positive HSPCs by 25-75% of the level before administration.

Optionally, the patient has a hematologic cancer treated by ablation of the HSPC's. Optionally, the patient is also administered an agent effective to treat the hematologic cancer. Optionally, the patient is administered the agent the before or during ablation of the HSPCs. Optionally, the agent is a chemotherapeutic agent, anti-angiogenic agent, anti-fibrotic agent or monoclonal antibody against a cancer antigen. Optionally, the hematologic cancer is a lymphoma, leukemia or myeloma. Optionally, the patient has a solid tumor and the patient is administered an agent effective to treat the solid tumor, and which damages HSPCs of the patient, before ablating the HSPCs in the patient. Optionally, the agent is a chemotherapeutic agent. Optionally, a CAR-T cell is administered to the patient after ablating the HSPCs. Optionally, a flt3 agonist or CISH inhibitor is administered after ablation of the HSPCs to promote growth of HSPCs or a cellular therapy. Optionally, an MCL1 inhibitor is co-administered with the immunotherapeutic agent specifically binding to c-kit and immunotherapeutic agent specifically binding to CD47 or SIRPα to ablate NK cells.

In any of the above methods, the patient can be a human.

The invention further provides for use of an immunotherapeutic agent specifically binding to c-kit in the manufacture of a medicament for ablating hematopoietic stem and progenitor cells (HSPCs), wherein the immunotherapeutic agent is for administration at a dose of 0.15-2 mg/kg in combination with an effective regime of an immunotherapeutic agent specifically binding to CD47 or SIRPα. The invention further provides for use of an immunotherapeutic agent specifically binding to CD47 or SIRPα in the manufacture of a medicament for ablating hematopoietic stem and progenitor cells (HSPCs) in combination with an immunotherapeutic agent specifically binding to c-kit at a dose of 0.15-2 mg/kg. Any of these uses can be in accordance with any method described above or herein.

### BRIEF DESCRIPTION OF THE FIGURES

FIGS. 1A, B show a treatment time line for administering (A) anti-c-kit and (B) anti-c-kit and anti-CD47 in primates.

FIG. 2 shows serum concentration of anti-c-kit with time.

FIG. 3 shows c-kit receptor occupancy for different dosages of anti-c-kit.

FIG. 4 shows anti-c-kit and anti-CD47 receptor occupancy with time.

FIGS. 5A, B shows % changes in c-kit positive HSCs relative to baseline treated with (A) anti-c-kit and (B) anti-c-kit and anti-CD47.

FIGS. 6A, B show changes in (A) peripheral white blood cells or (B) neutrophils over time with combination treatment with anti-c-kit and anti-CD47.

FIGS. 7A, B show (A) hemoglobin and (B) red blood cells levels over time for combined anti-c-kit anti-CD47 treatment relative to a negative control.

FIG. 8 show ablation of HSC's with a multi-dosing regime of both anti-c-kit and anti-CD47.

FIG. 9 shows ablation of HSPCs by anti-c-kit and anti-SIRPα.

### DEFINITIONS

A subject or patient includes both humans being treated by the disclosed methods and other animals, particularly mammals, including pets and laboratory animals, e.g. mice, rats, rabbits, and non-human primates. Thus the methods are applicable to both human therapy and veterinary applications.

An immunotherapeutic agent refers to an antibody or Fc-fusion protein against a designated target. For example antibodies against CD47 and a SIRPα-Fc fusion are immunotherapeutic agents against CD47.

Immunotherapeutic agents are typically provided in isolated form. This means that such an agent is typically at least 50% w/w pure of interfering proteins and other contaminants arising from its production or purification but does not exclude the possibility that the agent is combined with an excess of pharmaceutical acceptable carrier(s) or other vehicle intended to facilitate its use. Sometimes agents are at least 60, 70, 80, 90, 95 or 99% w/w pure of interfering proteins and contaminants from production or purification. Often an agent is the predominant macromolecular species remaining after its purification.

Specific binding of an immunotherapeutic agent to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once antibodies or other immunotherapeutic agents of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. In IgA, the heavy constant region is divided into CH1, CH2 and CH3. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, in bispecific the binding sites are different (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chain variable regions or between different light chain variable regions are assigned the same number. Although Kabat numbering can be used for antibody constant regions, the EU index is more commonly used, as is the case in this application.

The term “epitope” refers to a site on an antigen to which an arm of a bispecific antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20×. or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

C-kit is substantially saturated with anti-c-kit when increasing the dose of anti-c-kit administered to a subject does not increase the amount of anti-c-kit bound to c-kit by more than 10%.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcy receptors, particularly FcyRI and FcyRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term “antibody-dependent cellular phagocytosis,” or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

Unless otherwise apparent from the context reference to a range should be understood as also disclosing all subranges defined by integers within the range.

Any dosage or dosage range provided herein in mg/kg can be converted to an absolute dosage in mg using an exemplary human body weight of 70 kg, optionally with rounding of the dosage, or upper and lower bounds of the dosage to the nearest integer, or nearest 10, 50, 100, 500 or 1000 integer encompassing the calculated absolute dose. Thus, for example, a dosage range of 0.15-2 mg/kg can be converted to 10.5 to 140 mg, or with exemplary rounding, 10 to 150 mg. Likewise a dosage range of 10-30 mg/kg can be converted to 700-2100 mg, or with exemplary rounding 500-2500 mg.

### DETAILED DESCRIPTION

### I. General

The invention provides co-administration regimes of immunotherapeutic agents specifically binding to c-kit or inhibiting CD47-SIRPα for ablation of endogenous HSPCs. These regimes are based in part on the insight that delivery of relatively low levels of anti-c-kit results in saturation of binding to c-kit on HSPCs without significant reduction of the levels HSPCs. However, significant reduction of the level of HSPCs can be obtained when the action of anti-c-kit is promoted by an immunotherapeutic agent inhibiting CD47-SIRPα. Administration of sufficient anti-c-kit to achieve substantial saturation of receptors but not a vast excess is advantageous in reducing the level of HSPCs expressing c-kit to an acceptable level to permit introduction of replacement HSPCs without detrimental delay during which a subject has inadequate HSPCs.

### II. HSPCs

Depending on the application, the HSPCs to be introduced into a subject can be autologous (i.e., from that subject), allogenic (from another individual of the same species), or xenogenic (from a different species). If allogenic, the HSPCs can be matched fully or partially or unmatched for MHC alleles. Matched HSPCs can be obtained from a relative or a stranger.

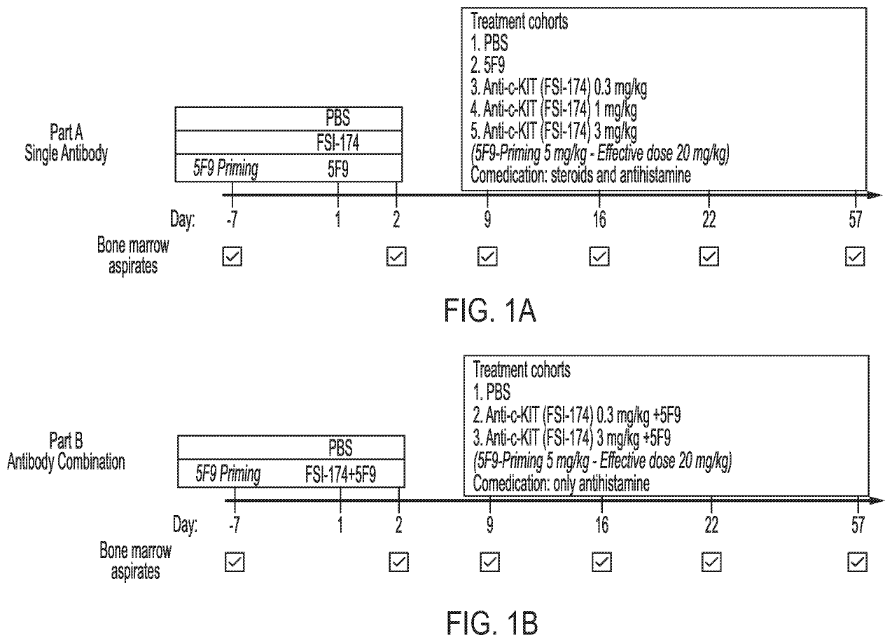

Although all HSPCs are capable of propagation and differentiation into cells of myeloid or lymphoid linages or both, HSPCs include cells at different stages of differentiation. Primitive stem cells can propagate indefinitely and form all cells types of myeloid and lymphoid lineages. Primitive stem cell differentiate into multi-potent progenitors, which can give rise to all cells of both myeloid and lymphoid lineages but cannot propagate indefinitely. Multipotent progenitors give rise to oligo-potent progenitors including the common lymphoid progenitor, CLP, which gives rise to mature B lymphocytes, T lymphocytes, and natural killer (NK) cells. Multipotent progenitors also give rise to the common myeloid progenitor (CMP) which further differentiates into granulocyte-macrophage progenitors, which differentiate into monocytes/macrophages and granulocytes, and megakaryocyte/erythrocyte progenitors, which differentiate into megakaryocytes/platelets and erythrocytes (see FIG. 1 of Bryder et al., Am. J. Pathol. 169, 338-346 (2006)).

Primitive hematopoietic stem cells (HC) and multipotent progenitor cells (HPC) can be distinguished from each other experimentally, for example, by performing a Cobblestone-Forming Area Cell Assay (Ploemacher et al. Blood. 78:2527-33 (1991)). Progenitor cells appear earlier, over a 1 to 3 week period in culture whereas the primitive hematopoietic stem cells appear at 4 to 5 weeks in culture. Both primitive stem cells and multipotent progenitor cells are useful for replacement therapy. Further differentiated cells such as the CMP or CLP can also be used but may be less versatile because of their limited propagation ability and restricted lineage of cells they are capable of forming.

HSPCs can be obtained by harvesting from bone marrow, from peripheral blood or umbilical cord blood. Bone marrow is generally aspirated from the posterior iliac crests while the donor is under either regional or general anesthesia. Additional bone marrow can be obtained from the anterior iliac crest. Bone marrow can be primed with granulocyte colony-stimulating factor (G-CSF; filgrastim [Neupogen]) to increase the stem cell count. Reference to “whole bone marrow” generally refers to a composition of mononuclear cells derived from bone marrow that have not been selected for specific immune cell subsets. “Fractionated bone marrow” may be, for example, depleted of T cells, e.g. CD8+ cells, CD52+ cells, CD3+ cells, etc.; enriched for CD34+ cells, and so forth.

HSPCs can also be obtained by mobilization of stem cells from the bone marrow into peripheral blood by cytokines such as G-CSF, GM-CSF or Plerixafor (also known as AMD3100 or Mozobil). An exemplary dose of G-CSF used for mobilization is 10 μg/kg/day but higher doses can be given up to e.g., 40 μg/kg/day can be given. Mozobil may be used in conjunction with G-CSF to mobilize HSPC to peripheral blood for collection. HSPCs can be harvested from peripheral blood with an apheresis device.

HSPCs can also be obtained from umbilical cord blood (UBC) typically for allogenic transplant. UCB is enriched in primitive stem/progenitor cells able to produce in vivo long-term repopulating stem cells.

Blood cells isolated from these procedures can undergo enrichment for HSPCs or a subset thereof, e.g., primitive stem cells and/or common progenitor by affinity enrichment for characteristic cell surface markers. Such markers include CD34; CD90 (thy-1); CD59; CD1 10 (c-mpl); c-kit (CD-117). Cells can be selected by affinity methods, including magnetic bead selection, flow cytometry, and the like from the donor hematopoietic cell sample. Several immunoselection devices, including Ceparte, Isolex 300i, and CliniMACS are commercially available for CD34+ cell selection.

The HSPC composition can be at least about 50% pure, as defined by the percentage of cells that are CD34+ in the population, may be at least about 75% pure, at least about 85% pure, at least about 95% pure, or more.

An exemplary market set characterizing HSPCs is CD34 positive, and lineage negative in each of CD11b, CD2, CD14, CD4, CD56, CD7, CD3, CD8a, CD16, CD19, CD20.

### III. Ablation Regimes

Ablation regimes serve to reduce or eliminate endogenous HSPCs. Endogenous HSPCs can be reduced by a factor of e.g., at least 10%, 25%, 50%, 75% or 90% before introducing replacement HSPCs. Some regimes do not reduce endogenous HSPCs by more than, e.g., 90%, 75%, 50%, 25% or 10% before introducing replacement HSPCs. Some regimes reduce endogenous HSPCs by 25-75% or 25-95%. Ablation regimes can also be defined by corresponding percentage reductions of HSCs or HPCs, the constituent cells of HSPCs. Because both express anti-c-kit, both can undergo ablation to a similar extent.

Such ablation regimes involve administration of an antibody specifically binding to c-kit (CD117) (see generally WO 2008067115) or other agent binding to an inhibitor c-kit, as exemplified further below. C-kit is also known as any of PBT, SCFR, MASTC. Human c-kit, which is what is targeted by immunotherapeutics in treatment of humans, has been assigned exemplary accession numbers NCBI Gene ID:3815 and Uniprot-P10721. C-kit is a cell surface marker used to identify certain types of HSPCs in the bone marrow. Hematopoietic stem cells (HSC), multipotent progenitors (MPP), and common myeloid progenitors (CMP) express high levels of c-kit. Such antibodies can reduce endogenous HSPCs by inhibiting interaction between c-kit and its ligand and by effector mediated mechanisms, such as ADCC, ADCP and CDC. c-kit is a receptor tyrosine kinase type III, which binds to stem cell factor (a substance that causes certain types of cells to grow), also known as “steel factor” or “c-kit ligand.” When this receptor binds to stem cell factor, it forms a dimer that activates its intrinsic tyrosine kinase activity, which in turn phosphorylates and activates signal transduction molecules that propagate the signal in the cell. A number of antibodies that specifically bind human c-kit are commercially available, including SR1, 2B8, ACK2, YB5-B8, 57A5, 104D2 (US20180214525). AMG 191 is a humanized form of SR1 (U.S. Pat. Nos. 8,436, 150, and 7,915,391). Any of these antibodies, including chimeric, veneered or humanized forms, or antibodies binding the same epitope or competing therewith for binding to c-kit can be used in the disclosed methods. Other antibodies against c-kit can be generated de novo by standard immunological techniques as further described below.

Some other humanized forms of SR1 are described by e.g., U.S.62/771,526 filed Nov. 26, 2018 and PCT/US2019/063091, filed Nov. 25, 2019. Some antibodies comprises heavy chains CDRs H1, H2 and H3 SEQ ID NOS. 2-4 respectively and light chain CDRs L1, L2 and L3 comprise SEQ ID NOS. 6-8 (i.e., as defined by Kabat) of U.S.62/771,526 and PCT/US2019/063091, optionally with one, two or three of the following CDR substitutions. These CDRs are SEQ ID NOS. 1-6 herein. The CDR substitution is preferably selected from N to A at heavy chain position 60, K to Q at heavy chain position 64 and N to Q at light chain position 30, positions being numbered according to Kabat. Some preferred antibodies of the invention have a mature heavy chain variable region having a sequence of any of the chains designated SEQ ID NOS. 13, 17 or 21 in U.S.62/771,526 and PCT/US2019/063091 and SEQ ID NOS. 7-9 herein, corresponding to AH2, AH3, and AH4 and a mature light chain variable region having a sequence of SEQ ID NO: 53 of U.S.62/771,526 and PCT/US2019/063091 (SEQ ID NO:10 herein) corresponding to NL2. In general, effector functions, such as ADCP, are useful although not essential for anti-c-kit antibodies. Therefore, some such antibodies have human IgG1 isotype. Some antibodies have human IgG1 isotype with an mutation to enhance one or more effector functions (see below). An exemplary antibody used in the Examples below is a humanized form of SR1 comprising a heavy chain variable region of SEQ ID NO:7 herein, and a light chain variable region of SEQ ID NO:10 herein and human IgG1 isotype.

Further examples of anti-c-kit immunotherapeutic agents include FSI-174 (Forty Seven, Inc.), and CDX-0158 or CDX-0159 (Celldex Therapeutics, Inc.) Other inhibitors of c-kit are described in the following publications: WO199203459, WO199221766, WO2007127317, WO2008115300, WO2012154480, WO2019155067, and WO2020076105.

The ablation regime can also include an immunotherapeutic agent inhibiting CD47-SIRPα interaction for use in combination with an antibody against c-kit (see generally WO2016033201). Such an agent promotes effector-mediated elimination of endogenous HSPCs mediated by anti-c-kit. Such agents include antibodies specifically binding to CD47 or SIRPα. Such agents also include a CD47 ECD fused to an Fc, which functions similarly to antibodies against SIRPα, or a SIRPα fused to an Fc, which functions similarly to antibodies against CD47. (See Zhang et al., Antibody Therapeutics, 1: 27-32 (2018)). Preferred antibodies antagonize CD47-SIRPα interaction without conferring an activating signal through either receptor.

CD47 is also known as any of IAP, MERG, and 0A3. Human CD47, which is targeted by immunotherapeutic agents in treatment of humans, has been assigned exemplary accession numbers NCBI Gene ID: 961 and UniProt Q08722.

Examples of suitable anti-CD47 antibodies include clones B6H12, 5F9, 8B6, C3, (for example as described in WO2011/143624) CC9002 (Vonderheide, Nat Med 2015; 21: 1122-3, 2015), and SRF231 (Surface Oncology). Suitable anti-CD47 antibodies include human, humanized or chimeric versions of such antibodies, antibodies binding to the same epitope or competing therewith for binding to CD47. Humanized antibodies (e.g., hu5F9-IgG4-WO2011/143624) are especially useful for in vivo applications in humans due to their low antigenicity. Direct contact residues for hu5F9-IgG4 in human CD47 have been reported to be K39, K41, E97, T99 and E104 (LC) and E29, R103 and E104 (HC) (Weiskopf et al., J. Clin. Invest 126, 2610-262-(2016)). Similarly caninized, felinized antibodies and the like are especially useful for applications in dogs, cats, and other species respectively.

Some humanized antibodies specifically binds to human CD47 comprising a variable heavy (VH) region containing the VH complementarity regions, CDR1, CDR2 and CDR3, respectively set forth in SEQ ID NO: 20, 21 and 22; and a variable light (VL) region containing the VL complementarity regions, CDR1, CDR2 and CDR3, respectively set forth in SEQ ID NO:23, 24 and 25 of WO2011/143624 (SEQ ID NOS:11-16 herein). Some humanized antibodies include a heavy chain variable region selected from SEQ ID NO: 36, SEQ ID NO: 37 and SEQ ID NO: 38 and a light chain variable region selected from SEQ ID NO: 41, SEQ ID NO: 42 and SEQ ID NO: 43 of WO2011/143624 (SEQ ID NOS. 17-22 herein). Magrolimab, a humanized form of 5F9, is a preferred antibody.

Other examples of immunotherapeutic agents against CD47 inhibiting its interaction with SIRPα include anti-CD47 mAbs (Vx-1004), anti-human CD47 mAbs (CNTO-7108), CC-90002, CC-90002-ST-001, NI-1701, NI-1801, RCT-1938, ALX-148, RRx-001, DSP-107, VT-1021, TTI-621, TTI-622, IMM-02 SGN-CD47M.

Suitable anti-SIRPα antibodies specifically bind SIRPα (without activating/stimulating enough of a signaling response to inhibit phagocytosis) and inhibit an interaction between SIRPα and CD47. Human SIRPα, which is targeted by immunotherapeutic agents in treatment of humans, has been assigned exemplary accession numbers NCBI Gene ID: 140885; and UniProt P78324. Suitable anti-SIRPα antibodies include fully human, humanized or chimeric versions of such antibodies. Some exemplary anti-SIRPα antibodies defined by their Kabat CDRs and variable regions are provided in Table 1 below.

TABLE 1
anti-SIRPα antibodies

| SEQ | | |
|---|---|---|
| ID NO | ID | Sequence |
| 23 | 1H9 | SYWIT |
| | CDR-H1 | |
| 24 | 1H9 | DIYPGSGSTNHIEKFKS |
| | CDR-H2 | |
| 25 | 1H9 | GYGSSYGYFDY |
| | CDR-H3 | |
| 26 | 1H9 | RASENIYSYLA |
| | CDR-L1 | |
| 27 | 1H9 | TAKTLAE |
| | CDR-L2 | |
| 28 | 1H9 | QHQYGPPFT |
| | CDR-L3 | |
| 29 | Humanized | QVQLVQSGAE VKKPGASVKV SCKASGYTFT SYWITWVKQA |
| | 1H9 V_{H} | PGQGLEWIGD IYPGSGSTNH IEKFKSKATL TVDTSISTAY |
| | | MELSRLRSDD TAVYYCATGY GSSYGYFDYW GQGTLVTVSS |
| 30 | Humanized | DIQMTQSPSS LSASVGDRVT ITCRASENIY SYLAWYQQKP |
| | 1H9 V_{L} | GKAPKLLIYT AKTLAEGVPS RFSGSGSGTD FTLTISSLQP |
| | | EDFATYYCQH QYGPPFTFGQ GTKLEIK |
| 31 | 3C2 | SYWMH |
| | CDR-H1 | |
| 32 | 3C2 | NIDPSDSDTHYNQKFKD |
| | CDR-H2 | |
| 33 | 3C2 | GYSKYYAMDY |
| | CDR-H3 | |
| 34 | 3C2 | RSSQSIVHSYGNTYLE |
| | CDR-L1 | |
| 35 | 3C2 | KVSNRFS |
| | CDR-L2 | |
| 36 | 3C2 | FQGSHVPYT |
| | CDR-L3 | |
| 37 | Humanized | QVQLVQSGAE VKKPGASVKV SCKASGYTFT SYWMHWVRQA |
| | 3C2 V_{H} | PGQGLEWMGN IDPSDSDTHY NQKFKDRVTM TRDTSTSTVY |
| | | MELSSLRSED TAVYYCARGY SKYYAMDYWG QGTLVTVSS |
| 38 | Humanized | DIVMTQTPLS LSVTPGQPAS ISCRSSQSIV HSYGNTYLEW |
| | 3C2 V_{L} | YLQKPGQSPQ LLIYKVSNRF SGVPDRFSGS GSGTDFTLKI |
| | | SRVEAEDVGV YYCFQGSHVP YTFGQGTKLE IK |
| 39 | Humanized | QVQLVQSGAEVKKPGASVKVSCKASGYTFTSYWITWVKQAPGQGLEWIG |
| | 1H9 HC | DIYPGSGSTNHIEKFKSKATLTVDTSISTAYMELSRLRSDDTAVYYCAT |
| | (full- | GYGSSYGYFDYWGQGTLVTVSSASTKGPSVFPLAPSSKSTSGGTAALGC |
| | length) | LVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFL |
| | | FPPKPKDTLMISRTPEVTCVVVDVSHEDPEVKFNWYVDGVEVHNAKTKP |
| | | REEQYASTYRVVSVLTVLHQDWLNGKEYKCKVSNKALPAPIEKTISKAK |
| | | GQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQ |
| | | KSLSLSPG |
| 40 | Humanized | DIQMTQSPSSLSASVGDRVTITCRASENIYSYLAWYQQKPGKAPKLLIY |
| | 1H9 LC | TAKTLAEGVPSRFSGSGSGTDFTLTISSLQPEDFATYYCQHQYGPPFTF |
| | (full- | GQGTKLEIKRTVAAPSVFIFPPSDEQLKSGTASVVCLLNNFYPREAKVQ |
| | length) | WKVDNALQSGNSQESVTEQDSKDSTYSLSSTLTLSKADYEKHKVYACEV |
| | | THQGLSSPVTKSFNRGEC |
| 41 | Humanized | QVQLVQSGAEVKKPGASVKVSCKASGYTFTSYWMHWVRQAPGQGLEWMG |
| | 3C2 HC | NIDPSDSDTHYNQKFKDRVTMTRDTSTSTVYMELSSLRSEDTAVYYCAR |
| | (full- | GYSKYYAMDYWGQGTLVTVSSASTKGPSVFPLAPSSKSTSGGTAALGCL |
| | length) | VKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPR |
| | | EEQYASTYRVVSVLTVLHQDWLNGKEYKCKVSNKALPAPIEKTISKAKG |
| | | QPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPG |
| 42 | Humanized | DIVMTQTPLSLSVTPGQPASISCRSSQSIVHSYGNTYLEWYLQKPGQSP |
| | 3C2 LC | QLLIYKVSNRFSGVPDRFSGSGSGTDFTLKISRVEAEDVGVYYCFQGSH |
| | (full- | VPYTFGQGTKLEIKRTVAAPSVFIFPPSDEQLKSGTASVVCLLNNFYPR |
| | length) | EAKVQWKVDNALQSGNSQESVTEQDSKDSTYSLSSTLTLSKADYEKHKV |
| | | YACEVTHQGLSSPVTKSFNRGEC |
| 43 | 9B11 | DYYIH |
| | CDR-H1 | |
| 44 | 9B11 | RIDPEDGETKYAPKFQG |
| | CDR-H2 | |
| 45 | 9B11 | GGFAY |
| | CDR-H3 | |
| 46 | 9B11 | ASSSVSSSYLY |
| | CDR-L1 | |
| 47 | 9B11 | STSNLAS |
| | CDR-L2 | |
| 48 | 9B11 | HQWSSHPYT |
| | CDR-L3 | |
| 49 | 9B11 V_{H} | EVQLQQSGAELVKPGASVKLSCTASGFNIKDYYIHWVKQRTEQGLEWIG |
| | | RIDPEDGETKYAPKFQGKATITADTSSNTAYLQLNSLTSEDTAVYSCAK |
| | | GGFAYWGQGTLVTVSA |
| 50 | 9B11 V_{L} | QIVLTQSPAIMSASPGEKVTLTCSASSSVSSSYLYWYQQKPGSSPKLWI |
| | | YSTSNLASGVPARFSGSGSGTSYSLTISSMEAEDAASYFCHQWSSHPYT |
| | | FGGGTKLEIK |
| 51 | 7E11 | SYWMH |
| | CDR-H1 | |
| 52 | 7E11 | NIDPSDSDTHYNQKFKD |
| | CDR-H2 | |
| 53 | 7E11 | SYGNYGENAMDY |
| | CDR-H3 | |
| 54 | 7E11 | RSSQSIVHSYGNTYLE |
| | CDR-L1 | |
| 55 | 7E11 | KVSNRFS |
| | CDR-L2 | |
| 56 | 7E11 | FQGSHVPFT |
| | CDR-L3 | |
| 57 | 7E11 V_{H} | QVKLQESGAELVRPGSSVKLSCKASGYTFTSYWMHWVKQRPIQGLEWIG |
| | | NIDPSDSDTHYNQKFKDKATLTVDNSSSTAYMQLSSLTSEDSAVYYCAS |
| | | YGNYGENAMDYWGQGTSVTVSS |
| 58 | 7E11 V_{L} | DILMTQTPLSLPVSLGDQASISCRSSQSIVHSYGNTYLEWYLQKPGQSP |
| | | KLLIYKVSNRFSGVPDRFSGSGSGTDFTLKISRVEAEDLGVYYCFQGSH |
| | | VPFTFGSGTKLEIK |

An exemplary antibody from the above table used in the Examples below is humanized 1H9 comprising a heavy chain variable region of SEQ ID NO:29 and light chain variable region of SEQ ID NO:30 and a human IgG1 constant region mutated for reduced effector function (N297A, EU numbering). Further exemplary antibodies are KWAR23 (Ring et al., Proc Natl Acad Sci U S A. 2017 Dec. 5; 114(49): E10578-E10585 , WO2015/138600), My-1 and Effi-DEM also known as B1765063 (Boehringer Ingelheim) (Zhang et al., Antibody Therapeutics, Volume 1, Issue 2, 21 Sep. 2018, Pages 27-32). Humanized antibodies are especially useful for in vivo applications in humans due to their low antigenicity. Similarly caninized, felinized, etc. antibodies are especially useful for applications in dogs, cats, and other species respectively. Other examples of anti-SIRPα-antibodies include FSI-189 (Forty Seven, Inc.), ES-004, ADU1805 (Aduro Biotech and, Voets et al, J Immunother. Cancer. 2019; 7: 340), and CC-95251 (Celgene, Uger & Johnson, Expert Opinion on Biological Therapy, 20:1, 5-8, DOI: 10.1080/14712598.2020.1685976).

Immunotherapeutic agents also include soluble CD47 polypeptides that specifically binds SIRPα and reduce the interaction between CD47 on an HSPC and SIRPα on a phagocytic cell (see, e.g., WO2016179399). Such polypeptides can include the entire ECD or a portion thereof with the above functionality. A suitable soluble CD47 polypeptide specifically binds SIRPα without activating or stimulating signaling through SIRPα because activation of SIRPα would inhibit phagocytosis. Instead, suitable soluble CD47 polypeptides facilitate the phagocytosis of endogenous HCSPs. A soluble CD47 polypeptide can be fused to an Fc (e.g., as described in US20100239579).

Other examples of agents binding to SIRPα and inhibiting its interaction with CD47 are described in WO200140307, WO2002092784, WO2007133811, WO2009046541, WO2010083253, WO2011076781, WO2013056352, WO2015138600, WO2016179399, WO2016205042, WO2017178653, WO2018026600, WO2018057669, WO2018107058, WO2018190719, WO2018210793, WO2019023347, WO2019042470, WO2019175218, WO2019183266, WO2020013170 and WO2020068752.

Immunotherapeutic reagents also include soluble SIRPα polypeptides specifically binding to CD47 and inhibiting its interaction with SIRPα. Exemplary agents include ALX148 (Kauder et al., Blood 2017 130:112) and TTI-622 and TTI-661 Trillium). Such agents can include the entire SIRPαECD or any portion thereof with the above functionality. The SIRPα reagent will usually comprise at least the dl domain of SIRPα. The soluble SIRPα polypeptide can be fused to an Fc region. Exemplary SIRPα polypeptides termed “high affinity SIRPα reagent”, which includes SIRPα-derived polypeptides and analogs thereof (e.g., CV1-hIgG4, and CV1 monomer are described in WO2013/109752. High affinity SIRPα reagents are variants of the native SIRPα protein. The amino acid changes that provide for increased affinity are localized in the dl domain, and thus high affinity SIRPα reagents comprise a dl domain of human SIRPα, with at least one amino acid change relative to the wild-type sequence within the dl domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, and so forth. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Immunotherapeutic agents directed at CD47 or SIRPα with an Fc region can have any of the human isotypes, e.g., IgG1, IgG2, IgG3 or IgG4. Human IgG4 or IgG2 isotype or IgG1 mutated to reduce effector functions can be used because effector functions are not required for inhibiting the CD47-SIRPα interaction.

Immunotherapeutic agents, including antibodies and Fc fusion proteins, are administered in a regime effective to achieve the desired purpose of reducing or eliminating endogenous HSPCs. An effective regime refers to a combination of dose, frequency of administration and route of administration. A regime of an antibody specifically binding to c-kit preferably of delivers sufficient antibody for substantial saturation of c-kit receptors on the target population of HSPC's expressing c-kit but not a vast excess over such an amount resulting in unnecessary long persistence of anti-c-kit after administration, which may result in delay in administering replacement HSPC's or unintended killing of replacement of HSPC's. It has been found that a total amount of anti-c-kit of about 0.15-2 mg/kg is suitable for such purposes. This amount can be administered as a single dose, two doses, or three or more dosages. One regime involves administering a single dose of e.g., 0.15-1 mg/kg, 0.25-1 mg/kg, 0.25-0.5 mg/kg, or 0.3 mg/kg anti-c-kit. Another regime involves administering two doses of e.g., 0.15-1 mg/kg, 0.25-1 mg/kg, 0.25-0.5 mg/kg, or 0.3 mg/kg anti-c-kit spaced by 3-7 days. Another regime involves administering three or more doses of 0.15-1 mg/kg, 0.25-1 mg/kg, 0.25-0.5 mg/kg, or 0.3 mg/kg anti-c-kit, optionally spaced by 10-30 days. Multiple administrations on the same days (i.e., within 24 hours first to last) are considered part of the same dose. Other regimes can deliver substantially the same areas of the curve (e.g., within 90%) of single, double or triple dosing regimes but with more administrations of reduced amounts on each administrations. Other regimes can result in substantially the same level of reduction of HSPCs expressing c-kit (e.g., within 90%) as the single and double dosing regimes described. Dosage provided are for antibodies to c-kit, particularly any of the humanized SR1 antibodies as described above. These dosages also provide guidance for other immunotherapeutic agents; however, dosages of such agents can be adjusted for differences in molecular weight and/or binding affinity to achieve substantially the same level of reduction of HSPCs expressing c-kit as obtained for humanized SR1.

Immunotherapy agents inhibiting CD47-SIRPα are administered in a regime effective to promote reduction of HSPCs expressing c-kit by an immunotherapeutic agent specifically binding to c-kit (anti-c-kit). Anti-c-kit may or may not effect reduction of HSPCs expressing c-kit in the absence promotion by an immunotherapy agent inhibiting CD47-SIRPα. Exemplary doses for immunotherapy agents inhibiting CD47-SIRPα are at least any of 0.05 mg/kg, 0.1 mg/kg, 0.5 mg/kg, 1 mg/kg up to any of 5 mg/kg, 10 mg/kg, 20 mg/kg, 30 mg/kg 40 mg/kg or 50 mg/kg. Some exemplary ranges are 0.05 mg/kg-50 mg/kg, 0. 1 mg/kg to 20 mg/kg, 1 mg/kg to 10 mg/kg, or 10 mg/kg to 30 mg/kg. Optionally, such immunotherapeutic agents, particularly those specifically binding to CD47, can be administered initially at one or more priming doses, followed by one or more therapeutic doses to reduce undesired crosslinking of red blood cells, as described by e.g., WO2017181033. A preferred regime is a priming dose at 0.5 to 5 mg/kg, e.g., 1 mg/kg following by a therapeutic dose of 10-30 or 15-20 mg/kg. The therapeutic dose is administered e.g., 3-15 or 5-10 or 7 days after the priming dose.

Combination or co-administration treatment with anti-c-kit and an immunotherapeutic agent inhibiting CD47-SIRPα involves administering the respective agents sufficiently proximal in time for the latter to promote reduction of HSPCs expressing c-kit by the former. Typically in combination regimes both agents are present at detectable levels in subject serum at the same time. In some combination regimes, a priming dose of an immunotherapeutic agent inhibiting CD47-SIRPα is administered followed by administration of a dose of anti-C-kit and a therapeutic dose of the immunotherapeutic agent inhibiting CD47-SIRPα at the same time. In some such regimes, the two agents are administered simultaneously by co-infusion. In some regimes, multiple doses of an immunotherapeutic agent specifically binding to c-kit and multiple doses of an immunotherapeutic agent inhibiting CD47-SIRPα are administered, optionally in a pairwise manner with one dose of each being administered on the same day. Such regimes can be preceded by a priming dose of the immunotherapeutic agent inhibiting CD47-SIRPα.

Combination treatment with an immunotherapeutic agent specifically binding to c-kit and an immunotherapeutic agent specifically binding to CD47 or SIRPα can be performed in further combination with one or more agents effective to deplete other cells of the immune system. For example, MCL1 apoptosis regulator, BCL2 family member (MCL1) inhibitors can be used to ablate NK cell. In various embodiments, an ablation regime as described herein, is combined with an inhibitor of MCL1 apoptosis regulator, BCL2 family member (MCL1, TM; EAT; MCL1L; MCL1S; Mcl-1; BCL2L3; MCL1-ES; bcl2-L-3; mcl1/EAT; NCBI Gene ID: 4170). Examples of MCL1 inhibitors include AMG-176, AMG-397, S-64315, AZD-5991, 483-LM, A-1210477, UMI-77, JKY-5-037, APG-3526 and those described in WO2018183418, WO2016033486, and WO2017147410.

Replacement HSPCs can be administered after administration of the combined regime of immunotherapeutic agents specifically binding to c-kit and inhibiting CD47-SIRPα. Optionally, replacement HSPCs are administered 5-15 days after administration of anti-c-kit or the last dose of anti-c-kit if more than one was administered. Administration can also be 5-15 days of the last dose of an immunotherapeutic agent inhibiting CD47-SIRPα if administered later than the last dose of anti-c-kit. Optionally levels of HSPCs or HSPCs expressing c-kit and/or levels of anti-c-kit and/or levels of the immunotherapeutic agent inhibiting CD47-SIRPα are measured and replacement HSPCs are administered when the level of HSPCs or HSPCs expressing c-kit falls below a threshold % of pretreatment levels (e.g., <90, 75, 50. 25 or 5% or 25-75% or 25-95%) and levels of anti-c-kit and/or the immunotherapeutic agent inhibiting CD47-SIRPα fall below 25, 10, 5, 1% of maximum levels or reach undetectable level.

Exemplary dosages of HSPCs for reintroduction are at least 1×10⁵, 1×10⁶, 2,×10⁶, 5×10⁶, 10⁷, 2×10⁷ CD34⁺ cells/kg body weight. Exemplary range are 1×10⁵ to 5×10⁷, 1×10⁶ to 2×10⁷, or 5×10⁵-6×10⁶ CD34⁺ cells/kg body weight. The dose may be limited by the number of available cells. Typically, regardless of the source, the dose is calculated by the number of CD34⁺ cells present. The percent number of CD34⁺ cells can be low for unfractionated bone marrow or mobilized peripheral blood; in which case the total number of cells administered is much higher. Component cells of HSPCs, such as HSC's or HPC's can be administered as an alternative to administered HSPC's.

Replacement HSPCs or other cellular therapies can be administered with one or more agent to promoter growth of HSPCs. For example, replacement HSPCs or other cellular therapies can be combined with administration an agonist of fms-related receptor tyrosine kinase 3 (FLT3); FLK2; STK1; CD135; FLK-2; NCBI Gene ID: 2322). Examples of FLT3 agonists include CDX-301 and GS-3583. Replacement HSPCs or other cellular therapies can also be combined with an inhibitor of cytokine inducible SH2 containing protein (CISH; CIS; G18; SOCS; CIS-1; BACTS2; NCBI Gene ID: 1154). Examples of CISH inhibitors include those described in WO2017100861, WO2018075664 and WO2019213610.

Immunotherapeutic agents are typically administered as pharmaceutical compositions in which the agent is combined with one or more pharmaceutically acceptable carriers. A variety of aqueous carriers can be used, e.g., buffered saline and the like. These solutions are sterile and generally free of undesirable matter. These compositions may be sterilized by conventional techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and is selected primarily based on fluid volumes, viscosities, body weight and the like in accordance with the particular mode of administration selected and the patient's needs (e.g., Remington's Pharmaceutical Science (15th ed. , 1980) and Goodman & Gillman, The Pharmacological Basis of Therapeutics (Hardman et al. , eds. , 1996)).

### IV. Cellular Therapies

In some embodiments, the cellular therapy entails co-administering immune cells engineered to express chimeric antigen receptors (CARs) or T cell receptors (TCRs) TCRs. In particular embodiments, a population of immune cells is engineered to express a CAR, wherein the CAR comprises a cancer antigen-binding domain. In other embodiments, a population of immune cells is engineered to express T cell receptors (TCRs) engineered to target tumor derived peptides presented on the surface of tumor cells. In one embodiment, the immune cell engineered to express chimeric antigen receptors (CARs) or T cell receptors (TCRs) TCRs is a T cell. In another embodiment, the immune cell engineered to express chimeric antigen receptors (CARs) or T cell receptors (TCRs) TCRs is an NK cell.

With respect to the structure of a CAR, in some embodiments, the CAR comprises an antigen binding domain, a transmembrane domain, and an intracellular signaling domain. In some embodiments, the intracellular domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCERIG), FcR beta (Fc Epsilon Rib), CD79a, CD79b, Fcgamma Rlla, DAP10, and DAP12 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, the costimulatory domain comprises a functional domain of one or more proteins selected from the group consisting of CD27, CD28, 4-1BB(CD137), OX40, CD30, CD40, PD-1, ICOS, CD2, CD7, LIGHT, NKG2C, lymphocyte function-associated antigen-1 (LFA-1), MYD88, B7-H3, a ligand that specifically binds with CD83, CDS, ICAM-1, GITR, BAFFR, HVEM (LIGHTR), SLAMF7, NKp80 (KLRFI), CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, ITGAE, CD103, ITGAL, CD1A (NCBI Gene ID: 909), CD1B (NCBI Gene ID: 910), CD1C (NCBI Gene ID: 911), CD1D (NCBI Gene ID: 912), CD1E (NCBI Gene ID: 913), ITGAM, ITGAX, ITGB1, CD29, ITGB2 (CD18, LFA-1), ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAM F1, CD150,IPO-3), BLAME (SLAMF8), SELPLG (CD162), LTBR, LAT, GADS, SLP-76, PAG/Cbp, NKp44, NKp30, NKp46, and NKG2D.

In some embodiments, the transmembrane domain comprises a transmembrane domain derived from a protein selected from the group consisting of the alpha, beta or zeta chain of the T-cell receptor, CD28, CD3 epsilon, CD3 delta, CD3 gamma, CD45, CD4, CDS, CD7, CD8 alpha, CD8 beta, CD9, CD11a, CD11b, CD11c, CD11d, CD16, CD18, CD22, CD33, CD37, CD64, CD80, CD86, CD134, CD137, CD154, KIRDS2, OX40, CD2, CD27, ICOS (CD278), 4-1BB(CD137), GITR, CD40, BAFFR, HVEM (LIGHTR), SLAMF7, NKp80 (KLRF1), CD19, CD19a, IL2R beta, IL2R gamma, IL7R alpha, ITGA1, VLA1, CD49a, ITGA4, IA4, CD49D, ITGA6, VLA-6, CD49f, ITGAD, CD1A, CD1B, CD1C, CD1D, CD1E, ITGAE, CD103, ITGAL, ITGAM, ITGAX, ITGB1, ITGB2, ITGB7, CD29, ITGB2 (LFA-1, CD18), ITGB7, TNFR2, DNAM1 (CD226), SLAMF4 (CD244, 2B4), CD84, CD96 (TACTILE), CEACAM1, CRTAM, Ly9 (CD229), CD160 (BY55), PSGL1, CD100 (SEMA4D), SLAMF6 (NTB-A, Ly108), SLAM (SLAMF1, CD150,IPO-3), BLAME (SLAMF8), SELPLG (CD162), LTBR, PAG/Cbp, NKp44, NKp30, NKp46, NKG2D, and NKG2C activating NK cell receptors, an Immunoglobulin protein, BTLA, CD247, CD276 (B7-H3), CD30, CD84, CDS, cytokine receptor, Fc gamma receptor, GADS, ICAM-1, Ig alpha (CD79a), integrins, LAT, a ligand that binds with CD83, LIGHT, MHC class 1 molecule, PAG/Cbp, TNFSF14, a Toll ligand receptor, TRANCE/RANKL, or a fragment, truncation, or a combination thereof.

In some embodiments, the CAR comprises a hinge domain. A hinge domain may be derived from a protein selected from the group consisting of the CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, IgG1, IgG2, IgG3, IgG4, IgA, IgD, IgE, IgM or fragment or combination thereof.

In some embodiments, the TCR or CAR antigen binding domain or the immunotherapeutic agent described herein (e.g., monospecific or multi-specific antibody or antigen-binding fragment thereof or antibody mimetic) binds a tumor-associated antigen (TAA). In some embodiments, the tumor-associated antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvIII); ganglioside G2 (GD2); ganglioside GD3 (αNeuSAc(2-8)αNeuSAc(2-3)βDGaip(1-4)bDGlcp(1-1)Cer); ganglioside GM3 (αNeuSAc(2-3)βDGalp(1-4)βDGlcp(1-1)Cer); GM-CSF receptor; TNF receptor superfamily member 17 (TNFRSF17, BCMA); B-lymphocyte cell adhesion molecule; Tn antigen ((Tn Ag) or (GaINAcu-Ser/Thr)); prostate-specific membrane antigen (PSMA); Receptor tyrosine kinase-like orphan receptor 1 (RORI); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); HLA class I antigen A-2 alpha; HLA antigen; Lewis(Y)antigen; CD24; Platelet-derived growth factor receptor beta (PDGFR-beta); Stage-specific embryonic antigen-4 (SSEA-4); CD20; delta like 3 (DLL3); Folate receptor alpha; Folate receptor beta, GDNF alpha 4 receptor, Receptor tyrosine-protein kinase, ERBB2 (Her2/neu); Mucin 1, cell surface associated (MUC1); APRIL receptor; ADP ribosyl cyclase-1; Ephb4 tyrosine kinase receptor, DCAMKL1 serine threonine kinase, Aspartate beta-hydroxylase, epidermal growth factor receptor (EGFR); neural cell adhesion molecule (NCAM); Prostase; prostatic acid phosphatase (PAP);elongation factor 2 mutated (ELF2M); Ephrin B2; fibroblast activation protein alpha (FAP);insulin-like growth factor 1 receptor (IGF-I receptor), carbonic anhydrase IX (CAIX);Proteasome (Prosome, Macropain) Subunit, Beta Type, 9 (LMP2); glycoprotein 100 (gp100);oncogene fusion protein consisting of breakpoint cluster region (BCR) and Abelson murine leukemia viral oncogene homolog 1 (Abl) (bcr-abl); tyrosinase; ephrin type-A receptor 2 (EphA2); ephrin type-A receptor 3 (EphA3), Fucosyl GM1; sialyl Lewis adhesion molecule (sLe); transglutaminase 5 (TGS5); high molecular weight-melanomaassociatedantigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); IL-15 receptor (IL-15); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma associated antigen 1 (MAGE-A1); Melanoma associated antigen 3 (MAGE-A3); Melanoma associated antigen 4 (MAGE-A4); T cell receptor beta 2 chain C; ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin-Al; Cyclin B1;v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES I); lymphocyte-specific protein tyrosine kinase (LCK); A kinase anchor protein 4 (AKAP-4); Peptidoglycan recognition protein, synovial sarcoma, X breakpoint 2 (SSX2); Receptor for Advanced Glycation End products (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-2 (GPC2); Glypican-3 (GPC3); Fc receptor-like 5 (FCRL5); and immunoglobulin lambda-like polypeptide 1 (IGLL1). In some embodiments, the target is an epitope of the tumor associated antigen presented in an MHC.

In some embodiments, the cancer antigen is selected from CD150, 5T4, ActRIIA, B7, TNF receptor superfamily member 17 (TNFRSF17, BCMA), CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD4OL, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, HER1-HER2 in combination, HER2-HER3 in combination, HERV-K, HIV-1 envelope glycoprotein gp120, HIV-1 envelope glycoprotein gp41, HLA-DR, HLA class I antigen alpha G, HM1.24, K-Ras GTPase, HMW-MAA, Her2, Her2/neu, IGF-1R, IL-11Ralpha, IL-13R-alpha2, IL-2, IL-22R-alpha, IL-6, IL-6R, la, li, L1-CAM, L1-cell adhesion molecule, Lewis Y, LI-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine e receptor, folate binding protein, gp100, hepatitis B surface antigen, Epstein-Barr nuclear antigen 1, Latent membrane protein 1, Secreted protein BARF1, P2X7 purinoceptor, Syndecan-1, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, P2-Microgiobuiin, Fc Receptor-like 5 (FcRL5).

Examples of cell therapies include without limitation: AMG-119, Algenpantucel-L, ALOFISEL®, Sipuleucel-T, (BPX-501) rivogenlecleucel US9089520, WO2016100236, AU-105, ACTR-087, activated allogeneic natural killer cells CNDO-109-AANK, MG-4101, AU-101, BPX-601, FATE-NK100, LFU-835 hematopoietic stem cells, Imilecleucel-T, baltaleucel-T, PNK-007, UCARTCS1, ET-1504, ET-1501, ET-1502, ET-190, CD19-ARTEMIS, ProHema, FT-1050-treated bone marrow stem cell therapy, CD4CARNK-92 cells, SNK-01, NEXI-001, CryoStim, AlloStim, lentiviral transduced huCART-meso cells, CART-22 cells, EGFRt/19-28z/4-1BBL CART cells, autologous 4H11-28z/fIL-12/EFGRt T cell, CCR5-SBC-728-HSPC, CAR4-1BBZ, CH-296, dnTGFbRII-NY-ESOc259T, Ad-RTS-IL-12, IMA-101, IMA-201, CARMA-0508, TT-18, CMD-501, CMD-503, CMD-504, CMD-502,CMD-601,CMD-602, CSG-005, LAAP T-cell therapy, PD-1 knockout T cell therapy (esophageal cancer/NSCLC), anti-MUC1 CAR T-cell therapy (esophageal cancer/NSCLC), anti-MUC1 CAR T-cell therapy+PD-1 knockout T cell therapy (esophageal cancer/NSCLC), anti-KRAS G12D mTCR PBL, anti-CD123 CAR T-cell therapy, anti-mutated neoantigen TCR T-cell therapy, tumor lysate/MUC1/survivin PepTivator-loaded dendritic cell vaccine, autologous dendritic cell vaccine (metastatic malignant melanoma, intradermal/intravenous), anti-LeY-scFv-CD28-zeta CAR T-cells, PRGN-3005, iC9-GD2-CAR-IL-15 T-cells, HSC-100, ATL-DC-101, MIDRIX4-LUNG, MIDRIXNEO, FCR-001, PLX stem cell therapy, MDR-101, GeniusVac-Mel4, ilixadencel, allogeneic mesenchymal stem cell therapy, romyelocel L, CYNK-001, ProTrans, ECT-100, MSCTRAIL, dilanubicel, FT-516, ASTVAC-2, E-CEL UVEC, CK-0801, allogenic alpha/beta CD3+ T cell and CD19+ B cell depleted stem cells (hematologic diseases, TBX-1400, HLCN-061, umbilical cord derived Hu-PHEC cells (hematological malignancies/aplastic anemia), AP-011, apceth-201, apceth-301, SENTI-101, stem cell therapy (pancreatic cancer), ICOVIR15-cBiTE, CD33HSC/CD33 CAR-T, PLX-Immune, SUBCUVAX, CRISPR allogeneic gamma-delta T-cell based gene therapy (cancer), ex vivo CRISPR allogeneic healthy donor NK-cell based gene therapy (cancer), ex-vivo allogeneic induced pluripotent stem cell-derived NK-cell based gene therapy (solid tumor), and anti-CD20 CAR T-cell therapy (non-Hodgkin's lymphoma),

### V. Co-Therapies for Treating Cancer

As previously described ablation of HSPC's can be used to treat hematopoietic cancers or to replace HSPC's damaged as a side effect of treatment of cancers of non-hematopoietic cells, e.g., solid tumors. Various examples of agents effective to treat cancer that can be used in combination with HSPC ablation are described below.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer.

Examples of chemotherapeutic agents include but not limited to: alkylating agents such as thiotepa and cyclophosphamide (CYTOXAN®); alkyl sulfonates such as busulfan, improsulfan, and piposulfan; aziridines such as benzodepa, carboquone, meturedepa, and uredepa; ethylenimines and methylamelamines including altretamine, triethylenemelamine, triethylenephosphoramide, triethylenethiophosphoramide, and trimemylolomelamine; acetogenins, e.g., bullatacin and bullatacinone; a camptothecin, including synthetic analog topotecan; bryostatin, callystatin; CC-1065, including its adozelesin, carzelesin, and bizelesin synthetic analogs; cryptophycins, particularly cryptophycin 1 and cryptophycin 8;dolastatin; duocarmycin, including the synthetic analogs KW-2189 and CBI-TMI; eleutherobin; 5-azacytidine; pancratistatin; a sarcodictyin; spongistatin; nitrogen mustards such as chlorambucil, chlornaphazine, cyclophosphamide, glufosfamide, evofosfamide, bendamustine, estramustine, ifosfamide, mechlorethamine, mechlorethamine oxide hydrochloride, melphalan, novembichin, phenesterine, prednimustine, trofosfamide, and uracil mustard; nitrosoureas such as carmustine, chlorozotocin, foremustine, lomustine, nimustine, and ranimustine; antibiotics such as the enediyne antibiotics (e.g., calicheamicin, especially calicheamicin gammall and calicheamicin phill), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin; anti-metabolites such as methotrexate and 5-fluorouracil (5-FU); folic acid analogs such as demopterin, methotrexate, pteropterin, and trimetrexate; purine analogs such as cladribine, pentostatin, fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replinishers such as frolinic acid; radiotherapeutic agents such as Radium-223, 177-Lu-PSMA-617 ; trichothecenes, especially T-2 toxin, verracurin A, roridin A, and anguidine; taxoids such as paclitaxel (TAXOL®), abraxane ,docetaxel (TAXOTERE®), cabazitaxel, BIND-014, tesetaxel; platinum analogs such as cisplatin and carboplatin, NC-6004 nanoplatin; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; trabectedin, triaziquone; 2,2′,2″-trichlorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; NUC-1031; FOLFOX (folinic acid, 5-fluorouracil, oxaliplatin); FOLFIRI (folinic acid, 5-fluorouracil, irinotecan); FOLFOXIRI (folinic acid, 5-fluorouracil, oxaliplatin, irinotecan), FOLFIRINOX (folinic acid, 5-fluorouracil, irinotecan, oxaliplatin), and pharmaceutically acceptable salts, acids, or derivatives of any of the above. Such agents can be conjugated onto an antibody or any targeting agent described herein to create an antibody-drug conjugate (ADC) or targeted drug conjugate.

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include apalutamide, abiraterone, enzalutamide, flutamide, galeterone, nilutamide, bicalutamide, leuprolide, goserelin, ODM-201, APC-100, ODM-204.

Additional examples of agents for targeting cancers include: alpha-fetoprotein modulators, such as ET-1402, and AFP-TCR; Anthrax toxin receptor 1 modulator, such as anti-TEM8 CAR T-cell therapy; TNF receptor superfamily member 17 (TNFRSF17, BCMA), such as bb-2121 (ide-cel), bb-21217, JCARH125, UCART-BCMA, ET-140, MCM-998, LCAR-B38M, CART-BCMA, SEA-BCMA, BB212, ET-140, P-BCMA-101, AUTO-2 (APRIL-CAR), JNJ-68284528;nti-CLL-1 antibodies, (see, for example, WO/2017/173384); Anti-PD-L1-CAR tank cell therapy, such as KD-045; Anti-PD-L1 t-haNK, such as PD-L1 t-haNK; anti-CD45 antibodies, such as 131I-BC8 (lomab-B);anti-HER3 antibodies, such as LJM716, GSK2849330; anti-CD52 antibodies, such as alemtuzumab; APRIL receptor modulator, such as anti-BCMA CAR T-cell therapy, Descartes-011;ADP ribosyl cyclase-1/APRIL receptor modulator, such as dual anti-BCMA/anti-CD38 CAR T-cell therapy; CART-ddBCMA;B7 homolog 6, such as CAR-NKp30 and CAR-B7H6; B-lymphocyte antigen CD19, such as TBI-1501, CTL-119 huCART-19 T cells, liso-cel, JCAR-015 U.S. Pat. No. 7,446,190, JCAR-014, JCAR-017, (WO2016196388, WO2016033570, WO2015157386), axicabtagene ciloleucel (KTE-C19, Yescarta®), KTE-X19, U.S. Pat. Nos. 7,741,465, 6,319,494, UCART-19, EBV-CTL, T tisagenlecleucel-T (CTL019), WO2012079000, WO2017049166, CD19CAR-CD28-CD3zeta-EGFRt-expressing T cells, CD19/4-1BBL armored CART cell therapy, C-CAR-011, CIK-CAR.CD19, CD19CAR-28-zeta T cells, PCAR-019, MatchCART, DSCAR-01, IM19 CAR-T, TC-110; anti-CD19 CAR T-cell therapy (B-cell acute lymphoblastic leukemia, Universiti Kebangsaan Malaysia); anti-CD19 CAR T-cell therapy (acute lymphoblastic leukemia/Non-Hodgkin's lymphoma, University Hospital Heidelberg), anti-CD19 CAR T-cell therapy (silenced IL-6 expression, cancer, Shanghai Unicar-Therapy Bio-medicine Technology), MB-CART2019.1 (CD19/CD20), GC-197 (CD19/CD7), CLIC-1901, ET-019003, anti-CD19-STAR-T cells, AVA-001, BCMA-CD19 cCAR (CD19/APRIL), ICG-134, ICG-132 (CD19/CD20), CTA-101, WZTL-002, dual anti-CD19/anti-CD20 CAR T-cells (chronic lymphocytic leukemia/B-cell lymphomas), HY-001, ET-019002, YTB-323, GC-012 (CD19/APRIL), GC-022 (CD19/CD22), CD19CAR-CD28-CD3zeta-EGFRt-expressing Tn/mem; UCAR-011, ICTCAR-014, GC-007F, PTG-01, CC-97540;Allogeneic anti-CD19 CART cells, such as GC-007G;APRIL receptor modulator; SLAM family member 7 modulator, BCMA-CS1 cCAR; Autologous dendritic cell tumor antigen (ADCTA), such as ADCTA-SSI-G; B-lymphocyte antigen CD20, such as ACTR707 ATTCK-20, PBCAR-20A; Allogenic T cells expressing CD20 CAR, such as LB-1905;B-lymphocyte antigen CD19/B-lymphocyte antigen 22, such as TC-310; B-lymphocyte antigen 22 cell adhesion, such as UCART-22, JCAR-018 WO2016090190; NY-ESO-1 modulators, such as GSK-3377794, TBI-1301, GSK3537142; Carbonic anhydrase, such as DC-Ad-GMCAIX; Caspase 9 suicide gene, such as CaspaClDe DLI, BPX-501; CCR5, such as SB-728; CCR5 gene inhibitor/TAT gene/TRIM5 gene stimulator, such as lentivirus vector CCR5 shRNA/TRIM5alpha/TAR decoy-transduced autologous CD34-positive hematopoietic progenitor cells; CDw123, such as MB-102, IM-23, JEZ-567, UCART-123; CD4, such as ICG-122; CD5 modulators, such as CD5.28z CART cells;Anti-CD22, such as anti-CD22 CART;Anti-CD30, such as TT-11; CD33, such as CIK-CAR.CD33, CD33CART;Dual anti-CD33/anti-CLL1, such as LB-1910; CD38, such as T-007, UCART-38;CD40 ligand, such as BPX-201, MED15083; CD56, such as allogeneic CD56-positive CD3-negative natural killer cells (myeloid malignancies); CD19/CD7 modulator, such as GC-197;T-cell antigen CD7 modulator, such as anti-CD7 CAR T-cell therapy (CD7-positive hematological malignancies); CD123 modulator, such as UniCAR02-T-CD123; Anti-CD276, such as anti-CD276 CART; CEACAM protein 5 modulators, such as MG7-CART; Claudin 6, such as CSG-002; Claudin 18.2, such as LB-1904;chlorotoxin, such as CLTX-CART;EBV targeted, such as CMD-003; MUC16EGFR, such as autologous 4H11-28z/fIL-12/EFGRt T cell; Endonuclease, such as PGN-514, PGN-201; Epstein-Barr virus specific T-lymphocytes, such as TT-10;Epstein-Barr nuclear antigen 1/Latent membrane protein 1/Secreted protein BARF1 modulator, such as TT-10X; Erbb2, such as CST-102, CIDeCAR; Ganglioside (GD2), such as 4SCAR-GD2; Gamma delta T cells, such as ICS-200; folate hydrolase 1 (FOLH1, Glutamate carboxypeptidase II, PSMA; NCBI Gene ID: 2346), such as CIK-CAR.PSMA, CART-PSMA-TGFβRDN, P-PSMA-101; Glypican-3(GPC3), such as TT-16, GLYCAR; hemoglobin, such as PGN-236; Hepatocyte growth factor receptor, such as anti-cMet RNA CAR T; HLA class I antigen A-2 alpha modulator, such as FH-MCVA2TCR; HLA class I antigen A-2 alpha/Melanoma associated antigen 4 modulator, such as ADP-A2M4CD8; HLA antigen modulator, such as FIT-001, NeoTCR-P1; Human papillomavirus E7 protein, such as KITE-439 (see, for example, WO/2015/184228); ICAM-1 modulator, such as AIC-100; Immunoglobulin gamma Fc receptor III, such as ACTR087; IL-12, such as DC-RTS-IL-12;IL-12 agonist/mucin 16, such as JCAR-020; IL-13 alpha 2, such as MB-101; IL-15 receptor agonist, such as PRGN-3006, ALT-803; interleukin-15/Fc fusion protein (e.g., XmAb24306); recombinant interleukin-15 (e.g., AM0015, NIZ-985); pegylated IL-15 (e.g., NKTR-255); IL-2, such as CST-101;Interferon alpha ligand, such as autologous tumor cell vaccine +systemic CpG-B+IFN-alpha (cancer);K-Ras GTPase, such as anti-KRAS G12V mTCR cell therapy; Neural cell adhesion molecule L1 L1CAM (CD171), such as JCAR-023; Latent membrane protein 1/Latent membrane protein 2, such as Ad5f35-LMPd1-2-transduced autologous dendritic cells; MART-1 melanoma antigen modulator, such as MART-1 F5 TCR engineered PBMC; Melanoma associated antigen 10, such as MAGE-A10C796T MAGE-A10 TCR; Melanoma associated antigen 3/ Melanoma associated antigen 6 (MAGE A3/A6) such as KITE-718 (see, for example, WO/2014/043441); Mesothelin, such as CSG-MESO, TC-210;Mucin 1 modulator, such as ICTCAR-052, Tn MUC-1 CAR-T, ICTCAR-053; Anti-MICA/MICB, such as CYAD-02; NKG2D, such as NKR-2; Ntrkr1 tyrosine kinase receptor, such as JCAR-024; PRAMET cell receptor, such as BPX-701;Prostate stem cell antigen modulator, such as MB-105;Roundabout homolog 1 modulator, such as ATCG-427; Peptidoglycan recognition protein modulator, such as Tag-7 gene modified autologous tumor cell vaccine; PSMA, such as PSMA-CAR T-cell therapy (lentiviral vector, castrate-resistant prostate cancer); SLAM family member 7 modulator, such as IC9-Luc90-CD828Z;TGF beta receptor modulator, such as DNR.NPC T-cells; T-lymphocyte, such as TT-12;T-lymphocyte stimulator, such as ATL-001;TSH receptor modulator, such as ICTCAR-051;Tumor infiltrating lymphocytes, such as LN-144, LN-145;and Wilms tumor protein, such as JTCR-016, WT1-CTL, or ASP-7517. An example progesterone receptor antagonist includes onapristone.

In various embodiments, an agent for treating cancer as described above, can be combined with an anti-angiogenic agent. Anti-angiogenic agents that can be co-administered include, but are not limited to, retinoid acid and derivatives thereof, 2-methoxyestradiol, ANGIOSTATIN®, ENDOSTATIN®, regorafenib, necuparanib, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs such as l-azetidine-2-carboxylic acid (LACA), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, metalloproteinase inhibitors such as BB-94, inhibitors of S100A9 such as tasquinimod . Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

In various embodiments, an agent for treating cancer as described above, is combined with an anti-fibrotic agent. Anti-fibrotic agents that can be co-administered include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. Nos. 5,021,456, 5,059,714, 5,120,764, 5,182,297, 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

Some chemotherapy agents are suitable for treating lymphoma or leukemia. These agents include aldesleukin, alvocidib, amifostine trihydrate, aminocamptothecin, antineoplaston A10, antineoplaston AS2-1, anti-thymocyte globulin, arsenic trioxide, Bcl-2 family protein inhibitor ABT-263, beta alethine, BMS-345541bortezomib (VELCADE®, PS-341), bryostatin 1, bulsulfan, campath-1H, carboplatin, carfilzomib (Kyprolis®), carmustine, caspofungin acetate, CC-5103, chlorambucil, CHOP (cyclophosphamide, doxorubicin, vincristine, and prednisone), cisplatin, cladribine, clofarabine, curcumin, CVP (cyclophosphamide, vincristine, and prednisone), cyclophosphamide, cyclosporine, cytarabine, denileukin diftitox, dexamethasone, docetaxel, dolastatin 10, doxorubicin, doxorubicin hydrochloride, DT-PACE (dexamethasone, thalidomide, cisplatin, doxorubicin, cyclophosphamide, and etoposide), enzastaurin, epoetin alfa, etoposide, everolimus (RAD001), FCM (fludarabine, cyclophosphamide, and mitoxantrone), FCR (fludarabine, cyclophosphamide, and rituximab), fenretinide, filgrastim, flavopiridol, fludarabine, FR (fludarabine and rituximab), geldanamycin (17 AAG), hyperCVAD (hyperfractionated cyclophosphamide, vincristine, doxorubicin, dexamethasone, methotrexate, and cytarabine), ICE (iphosphamide, carboplatin, and etoposide), ifosfamide, irinotecan hydrochloride, interferon alpha-2b, ixabepilone, lenalidomide (REVLIMID®, CC-5013), pomalidomide (POMALYST®/IMNOVID®)lymphokine-activated killer cells, MCP (mitoxantrone, chlorambucil, and prednisolone), melphalan, mesna, methotrexate, mitoxantrone hydrochloride, motexafin gadolinium, mycophenolate mofetil, nelarabine, obatoclax (GX15-070), oblimersen, octreotide acetate, omega-3 fatty acids, Omr-IgG-am (WNIG, Omrix), oxaliplatin, paclitaxel, palbociclib (PD0332991), pegfilgrastim, PEGylated liposomal doxorubicin hydrochloride, perifosin, prednisolone, prednisone, recombinant flt3 ligand, recombinant human thrombopoietin, recombinant interferon alfa, recombinant interleukin-11, recombinant interleukin-12, rituximab, R-CHOP (rituximab and CHOP), R-CVP (rituximab and CVP), R-FCM (rituximab and FCM), R-ICE (rituximab and ICE), and R MCP (rituximab and MCP), R-roscovitine (seliciclib, CYC202), sargramostim, sildenafil citrate, simvastatin, sirolimus, styryl sulphones, tacrolimus, tanespimycin, temsirolimus (CCI-779), thalidomide, therapeutic allogeneic lymphocytes, thiotepa, tipifarnib, vincristine, vincristine sulfate, vinorelbine ditartrate, SAHA (suberanilohydroxamic acid, or suberoyl, anilide, and hydroxamic acid), vemurafenib (Zelboraf ®), venetoclax (ABT-199).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

Treatment of non-Hodgkin's lymphomas (NHL), especially those of B cell origin, includes using monoclonal antibodies, standard chemotherapy approaches (e.g., CHOP (cyclophosphamide, doxorubicin, vincristine, and prednisone), CVP (cyclophosphamide, vincristine, and prednisone), FCM (fludarabine, cyclophosphamide, and mitoxantrone), MCP (Mitoxantrone, Chlorambucil, Prednisolone), all optionally including rituximab (R) and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP (rituximab, cyclophosphamide, doxorubicin, vincristine, and prednisone), R-FCM, R-CVP, and R MCP.

Examples of radioimmunotherapy for NHL/B-cell cancers include yttrium-90 ibritumomab tiuxetan (ZEVALIN®) and iodine-131 tositumomab (BEXXAR®).

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering anti-angiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are sirolimus, temsirolimus (TORISEL®, CCI-779), CC-115, CC-223, SF-1126, PQR-309 (bimiralisib), voxtalisib, GSK-2126458, and temsirolimus in combination with RITUXAN®, VELCADE®, or other chemotherapeutic agents.

Other recent therapies for MCL have been disclosed. Such examples include flavopiridol, palbociclib (PD0332991), R-roscovitine (selicicilib, CYC202), styryl sulphones, obatoclax (GX15-070), TRAIL, Anti-TRAIL death receptors DR4 and DR5 antibodies, temsirolimus (TORISEL®, CCI-779), everolimus (RAD001), BMS-345541, curcumin, SAHA, thalidomide, lenalidomide (REVLIMID®, CC-5013), and geldanamycin (17 AAG).

Therapeutic agents used to treat Waldenstrom's Macroglobulinemia (WM) include aldesleukin, alemtuzumab, alvocidib, amifostine trihydrate, aminocamptothecin, antineoplaston A10, antineoplaston AS2-1, anti-thymocyte globulin, arsenic trioxide, autologous human tumor-derived HSPPC-96, Bcl-2 family protein inhibitor ABT-263, beta alethine, bortezomib (VELCADE®), bryostatin 1, busulfan, campath-1H, carboplatin, carmustine, caspofungin acetate, CC-5103, cisplatin, clofarabine, cyclophosphamide, cyclosporine, cytarabine, denileukin diftitox, dexamethasone, docetaxel, dolastatin 10, doxorubicin hydrochloride, DT-PACE, enzastaurin, epoetin alfa, epratuzumab (hLL2-anti-CD22 humanized antibody), etoposide, everolimus, fenretinide, filgrastim, fludarabine, ibrutinib, ifosfamide, indium-111 monoclonal antibody MN-14, iodine-131 tositumomab, irinotecan hydrochloride, ixabepilone, lymphokine-activated killer cells, melphalan, mesna, methotrexate, mitoxantrone hydrochloride, monoclonal antibody CD19 (such as tisagenlecleucel-T, CART-19, CTL-019), monoclonal antibody CD20, motexafin gadolinium, mycophenolate mofetil, nelarabine, oblimersen, octreotide acetate, omega-3 fatty acids, oxaliplatin, paclitaxel, pegfilgrastim, PEGylated liposomal doxorubicin hydrochloride, pentostatin, perifosine, prednisone, recombinant flt3 ligand, recombinant human thrombopoietin, recombinant interferon alfa, recombinant interleukin-11, recombinant interleukin-12, rituximab, sargramostim, sildenafil citrate (VIAGRA®), simvastatin, sirolimus, tacrolimus, tanespimycin, thalidomide, therapeutic allogeneic lymphocytes, thiotepa, tipifarnib, tositumomab, ulocuplumab, veltuzumab, vincristine sulfate, vinorelbine ditartrate, vorinostat, WT1 126-134 peptide vaccine, WT-1 analog peptide vaccine, yttrium-90 ibritumomab tiuxetan, yttrium-90 humanized epratuzumab, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R ICE.

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

Myelofibrosis inhibiting agents include, but are not limited to, hedgehog inhibitors, histone deacetylase (HDAC) inhibitors, and tyrosine kinase inhibitors. Non-limiting examples of hedgehog inhibitors are saridegib and vismodegib. Examples of HDAC inhibitors include, but are not limited to, pracinostat and panobinostat. Non-limiting examples of tyrosine kinase inhibitors are lestaurtinib, bosutinib, imatinib, radotinib, and cabozantinib.

Gemcitabine, nab-paclitaxel, and gemcitabine/nab-paclitaxel may be used with a JAK inhibitor and/or PI3Kδ inhibitor to treat hyperproliferative disorders.

Therapeutic agents used to treat bladder cancer include atezolizumab, carboplatin, cisplatin, docetaxel, doxorubicin, fluorouracil (5-FU), gemcitabine, idosfamide, Interferon alfa-2b, methotrexate, mitomycin, nab-paclitaxel, paclitaxel, pemetrexed, thiotepa, vinblastine , and any combination thereof.

Therapeutic agents used to treat breast cancer include albumin-bound paclitaxel, anastrozole, capecitabine, carboplatin, cisplatin, cyclophosphamide, docetaxel, doxorubicin, epirubicin, everolimus, exemestane, fluorouracil, fulvestrant, gemcitabine, Ixabepilone, lapatinib, Letrozole, methotrexate, mitoxantrone, paclitaxel, pegylated liposomal doxorubicin, pertuzumab, tamoxifen, toremifene, trastuzumab, vinorelbine, and any combinations thereof.

Therapeutic agents used to treat triple negative breast cancer include cyclophosphamide, docetaxel, doxorubicin, epirubicin, fluorouracil, paclitaxel, and combinations thereof.

Therapeutic agents used to treat colorectal cancer include bevacizumab, capecitabine, cetuximab, fluorouracil, irinotecan, leucovorin, oxaliplatin, panitumumab, ziv-aflibercept, and any combinations thereof.

Therapeutic agents used to treat castration-resistant prostate cancer include abiraterone, cabazitaxel, docetaxel, enzalutamide, prednisone, sipuleucel-T, and any combinations thereof.

Therapeutic agents used to treat esophageal and esophagogastric junction cancer include capecitabine, carboplatin, cisplatin, docetaxel, epirubicin, fluoropyrimidine, fluorouracil, irinotecan, leucovorin, oxaliplatin, paclitaxel, ramucirumab, trastuzumab, and any combinations thereof.

Therapeutic agents used to treat gastric cancer include capecitabine, carboplatin, cisplatin, docetaxel, epirubicin, fluoropyrimidine, fluorouracil, Irinotecan, leucovorin, mitomycin, oxaliplatin, paclitaxel, ramucirumab, trastuzumab, and any combinations thereof.

Therapeutic agents used to treat head & neck cancer include afatinib, bleomycin, capecitabine, carboplatin, cetuximab, cisplatin, docetaxel, fluorouracil, gemcitabine, hydroxyurea, methotrexate, nivolumab, paclitaxel, pembrolizumab, vinorelbine, and any combinations thereof.

Therapeutic agents used to treat hepatobiliary cancer include capecitabine, cisplatin, fluoropyrimidine, 5-fluorourcil, gemcitabine, oxaliplatin, sorafenib, and any combinations thereof.

Therapeutic agents used to treat hepatocellular carcinoma include capecitabine, doxorubicin, gemcitabine, sorafenib, and any combinations thereof.

Therapeutic agents used to treat non-small cell lung cancer (NSCLC) include afatinib, albumin-bound paclitaxel, alectinib, bevacizumab, bevacizumab biosimilar, cabozantinib, carboplatin, cisplatin, crizotinib, dabrafenib, docetaxel, erlotinib, etoposide, gemcitabine, nivolumab, paclitaxel, pembrolizumab, pemetrexed, ramucirumab, trametinib, trastuzumab, vandetanib, vemurafenib, vinblastine, vinorelbine, and any combinations thereof.

Therapeutic agents used to treat small cell lung cancer (SCLC) include bendamustime, carboplatin, cisplatin, cyclophosphamide, docetaxel, doxorubicin, etoposide, gemcitabine, ipillimumab, irinotecan, nivolumab, paclitaxel, temozolomide, topotecan, vincristine, vinorelbine, and any combinations thereof.

Therapeutic agents used to treat melanoma cancer include albumin bound paclitaxel, carboplatin, cisplatin, cobiemtinib, dabrafenib, dacrabazine, IL-2, imatinib, interferon alfa-2b, ipilimumab, nitrosourea, nivolumab, paclitaxel, pembrolizumab, pilimumab, temozolomide, trametinib, vemurafenib, vinblastine, and any combinations thereof.

Therapeutic agents used to treat ovarian cancer include 5-flourouracil, albumin bound paclitaxel, altretamine, anastrozole, bevacizumab, capecitabine, carboplatin, cisplatin, cyclophosphamide, docetaxel, doxorubicin, etoposide, exemestane, gemcitabine, ifosfamide, irinotecan, letrozole, leuprolide acetate, liposomal doxorubicin, megestrol acetate, melphalan, olaparib, oxaliplatin, paclitaxel, Pazopanib, pemetrexed, tamoxifen, topotecan, vinorelbine, and any combinations thereof.

Therapeutic agents used to treat pancreatic cancer include 5-fluorourcil, albumin-bound paclitaxel, capecitabine, cisplatin, docetaxel, erlotinib, fluoropyrimidine, gemcitabine, irinotecan, leucovorin, oxaliplatin, paclitaxel, and any combinations thereof.

Therapeutic agents used to treat renal cell carcinoma include axitinib, bevacizumab, cabozantinib, erlotinib, everolimus, levantinib, nivolumab, pazopanib, sorafenib, sunitinib, temsirolimus, and any combinations thereof.

### VI. General Characteristics of Antibodies

The production of other non-human monoclonal antibodies, e.g., murine, guinea pig, primate, rabbit or rat, against an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

Antibodies are screened for specific binding to their intended target. Antibodies may be further screened for binding to a specific region of the target (e.g., containing a desired epitope), competition with a reference antibody, agonism or antagonism of cells bearing the antigen. Non-human antibodies can be converted to chimeric, veneered or humanized forms as described above.

The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428, S or N at position 434, Y at position 252, Tat position 254, and E at position 256. N434A (EU numbering). Increased FcRn binding is advantageous in making the hybrid proteins of the present invention compete more strongly with endogenous IgG for binding to FcRn. Also numerous mutations are known for reducing any of ADCC, ADP or CMC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution any of positions 234, 235, 236 and/or 237 reduce affinity for Fcy receptors, particularly FcyRI receptor (see, e.g., U.S. Pat. No. 6,624,821). Optionally, positions 234, 236 and/or 237 in human IgG2 are substituted with alanine and position 235 with glutamine or glutamic acid. (See, e.g., U.S. Pat. No. 5,624,821.) Other substitutions reducing effector function include A at position 268, G or A at position 297, L at position 309, A at position 322, G at position 327, S at position 330, S at position 331, S at position 238, A at position 268, L at position 309. Some examples of mutations enhancing effector function include S239D, I332E, A330L and combinations thereof.

As noted, In some embodiments, the Fc region of an antibody comprise one or more amino acid modifications that promote an increased serum half-life of the anti-binding molecule. Mutations that increase the half-life of an antibody have been described. In one embodiment, the Fc region or Fc domain of one or both of the CD3-targeting heavy chain and the HIV antigen-targeting heavy chain comprise a methionine to tyrosine substitution at position 252 (EU numbering), a serine to threonine substitution at position 254 (EU numbering), and a threonine to glutamic acid substitution at position 256 (EU numbering). See, e.g., U.S. Pat. No. 7,658,921. This type of mutant, designated as a “YTE mutant” exhibits a four-fold increased half-life relative to wild-type versions of the same antibody (Dall' Acqua, et al., J Biol Chem, 281: 23514-24 (2006); Robbie, et al., Antimicrob. Agents Chemotherap., 57(12):6147-6153 (2013)). In certain embodiments, the Fc region or Fc domain of one or both of the CD3-targeting heavy chain and the HIV antigen-targeting heavy chain comprise an IgG constant domain comprising one, two, three or more amino acid substitutions of amino acid residues at positions 251-257, 285-290, 308-314, 385-389, and 428-436 (EU numbering). Alternatively, M428L and N434S (“LS”) substitutions can increase the pharmacokinetic half-life of the multi-specific antigen binding molecule. In other embodiments, the Fc region comprises a M428L and N434S substitution (EU numbering). In other embodiments, the Fc region or Fc domain of one or both of the CD3-targeting heavy chain and the HIV antigen-targeting heavy chain comprise T250Q and M428L (EU numbering) mutations. In other embodiments, the Fc region comprise H433K and N434F (EU numbering) mutations.

As noted, the Fc region of an antibody can include post-translational and/or amino acid modifications that increase effector activity, e.g., have improved FcyIIIa binding and increased antibody-dependent cellular cytotoxicity (ADCC). In some embodiments, the Fc region or Fc domain of the antibody comprises DE modifications (i.e., S239D and I332E by EU numbering) in the Fc region. In some embodiments, the Fc region or Fc domain of the antibody comprises DEL modifications (i.e., S239D, I332E and A330L by EU numbering) in the Fc region. In some embodiments, the Fc region or Fc domain of the antibody comprises DEA modifications (i.e., S239D, I332E and G236A by EU numbering) in the Fc region. In some embodiments, the Fc region or Fc domain of the antibody comprises DEAL modifications (i.e., S239D, I332E, G236A and A330L by EU numbering) in the Fc region. See, e.g., U.S. Pat. Nos. 7,317,091; 7,662,925; 8,039,592; 8,093,357; 8,093,359; 8,383,109; 8,388,955; 8,735,545; 8,858,937; 8,937,158; 9,040,041; 9,353,187; 10,184,000; and 10,584,176. Additional amino acid modifications that increase effector activity, e.g., have improved Fcyllla binding and increased antibody-dependent cellular cytotoxicity (ADCC) include without limitation (EU numbering) F243L/R292P/Y300L/V305I/P396L; S298A/E333A/K334A; or L234Y/L235Q/G236W/S239M/H268D/D270E/S298A on a first Fc domain and D270E/K326D/A330M/K334E on a second Fc domain. Amino acid mutations that increase C1q binding and complement-dependent cytotoxicity (CDC) include without limitation (EU numbering) S267E/H268F/S324T or K326W/E333S. Fc region mutations that enhance effector activity are reviewed in, e.g., Wang, et al., Protein Cell (2018) 9(1): 63-73; and Saunders, Front Immunol. (2019) 10:1296.

In other embodiments, the antibody or antigen-binding fragment thereof has modified glycosylation, which, e.g., may be introduced post-translationally or through genetic engineering. In some embodiments, the antibody or antigen-binding fragment thereof is afucosylated, e.g., at a glycosylation site present in the antibody or antigen-binding fragment thereof. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcyR family. Afucosylated monoclonal antibodies are monoclonal antibodies engineered so that the oligosaccharides in the Fc region of the antibody do not have any fucose sugar units.

Antibodies of interest for ablation may be tested for their ability to induce ADCC. Antibody-associate ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-1 1-dUTP nick end labeling (TUNEL) assay (Lazebnik et al., Nature: 371 , 346 (1994). Cytotoxicity may also be detected directly by detection kits, such as Cytotoxicity Detection Kit from Roche Applied Science (Indianapolis, Ind.). Antibodies can likewise be tested for their ability to induce antibody dependent phagocytosis (ADP) on for example AML LSC as described by WO/2009/091601.

In some embodiments, an immunotherapeutic agent is conjugated to an effector moiety. The effector moiety can be any number of molecules, including labeling moieties such as radioactive labels or fluorescent labels, or can be a cytotoxic moiety. Cytotoxic agents include cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, saporin, auristatin-E and the like. Cytotoxic agents also include radiochemicals made by conjugating radioisotopes to antibodies. Targeting the cytotoxic moiety to transmembrane proteins serves to increase the local concentration of the cytotoxic moiety in the targeted area.

### VII. Genetic Disorders of Blood Cells

The present methods can be used to correct genetic disorders of blood cells, particularly monogenic disorders arising from mutation of a single protein. Such disorders can be dominant or non-dominant and may result in partial or complete penetrance. In general such disorders can be treated by ablating endogenous HPLC and administering replacement HPLCs which include a functioning (e.g., wildtype) form of the protein underlying the disorder. Such cells can express the wildtype protein as well as or instead of the mutant form of the protein depending on how the genetic modification is carried out.

Genetic disorders of blood cells include hemoglobinopathies, such as thalassemia's and sickle cell disease, X-linked severe combined immunodeficiency (X-SCID) adenosine deaminase deficiency (ADA-SCID), other genetic forms of SCID (artemis, Rag1/2), Wiskott Aldrich syndrome (WAS) , chronic granulomatous disease, hemophagocytic lymphohistiocytosis, X-linked hyper IgM syndrome, X-linked lymphoproliferative disease, X-linked agammaglobulinemia, X-linked adrenoleukodystrophy, metachromatic leukodystrophy, hemophilia, von Willebrand disease, drepanocytic anemia, hereditary aplastic anemia, pure red cell aplasia, paroxysmal nocturnal hemoglobinuria, Fanconi anemia, hemophagocytic lymphohistiocytosis (HLH), inborn errors of metabolism, e.g., mucopolysaccharidosis, Gaucher disease and other lipidoses, epidermolysis bullosa, severe congenital neutropenia, Shwachman-Diamond syndrome, Diamond-Blackfan anemia, Kostmann's syndrome and leukocyte adhesion deficiency.

In sickle cells anemia, valine is substituted for glutamic acid in the sixth amino acid of the hemoglobin beta chain. The valine mutant form of hemoglobin is much less soluble than the glutamic form; it forms a semisolid gel of rodlike factoids that cause RBCs to sickle at sites of low P02. Distorted, inflexible RBCs adhere to vascular endothelium and plug small arterioles and capillaries, which leads to occlusion and infarction. Because sickled RBCs are too fragile to withstand the mechanical trauma of circulation, hemolysis occurs after they enter the circulation. In homozygotes, clinical manifestations are caused by anemia and vaso-occlusive events resulting in tissue ischemia and infarction. Growth and development are impaired, and susceptibility to infection increases. Anemia is usually severe but varies highly among patients. Sick cell anemia can be remedied by correcting the genetic defect, expressing an additional functional hemoglobin transcriptional unit or disruption of the BCL11A erythroid enhance, which represses fetal globin expression resulting in increased levels of fetal hemoglobin for treatment of sickle cell anemia (or beta thalassemia).

Thalassemias are a group of chronic, inherited, microcytic anemias characterized by defective hemoglobin synthesis and ineffective erythropoiesis, particularly common in persons of Mediterranean, African, and Southeast Asian ancestry. Thalassemia is among the most common inherited hemolytic disorders. It results from unbalanced Hb synthesis caused by decreased production of at least one globin polypeptide chain (β, α, γ, δ). This can occur through mutations in the regulatory regions of the genes or from a mutation in a globin coding sequence that results in reduced expression.

Combined immunodeficiency is a group of disorders characterized by congenital and usually hereditary deficiency of both B- and T-cell systems, lymphoid aplasia, and thymic dysplasia. The combined immunodeficiencies include severe combined immunodeficiency, Swiss agammaglobulinemia, combined immunodeficiency with adenosine deaminase or nucleoside phosphorylase deficiency, and combined immunodeficiency with immunoglobulins (Nezelof syndrome). Most patients have an early onset of infection with thrush, pneumonia, and diarrhea. If left untreated, most die before age 2. Most patients have profound deficiency of B cells and immunoglobulin. The following are characteristic: lymphopenia, low or absent T-cell levels, poor proliferative response to mitogens, cutaneous anergy, an absent thymic shadow, and diminished lymphoid tissue. Pneumocystis pneumonia and other opportunistic infections are common.

The present methods can also be used be used for treatment of infectious disease by modifying an immune cell receptor used by infecting viruses, such as CCR5 in the case of HIV.

These present methods can also be used to treat hematologic malignancies and autoimmune diseases in which the pathology at least in part resides in blood cells. Hematologic malignancies include leukemia, lymphomas and myelomas. More specific examples of such malignancies include multiple myeloma, Non-Hodgkin lymphoma, Hodgkin disease, acute myeloid leukemia, acute lymphoid leukemia, acute lymphoblastic leukemia, chronic myeloid leukemia; chronic lymphocytic leukemia, myeloproliferative disorders, and multiple myeloma. Autoimmune disorders include B and T-cell mediated disorders. Common examples are rheumatoid arthritis, systemic lupus erythematosus, inflammatory bowel disease, multiple sclerosis, type 1 diabetes, Guillain Barre syndrome, chronic inflammatory demyelinating polyneuropathy, psoriasis, Grave's disease, Hasimoto's thyroiditis, myasthenia gravis, vasculitis and systemic sclerosis.

The present methods can also be used for replacing endogenous HSPCs in patients with other types of cancer, such as solid tumors, who have received chemotherapy causing damage to endogenous HSPCs. Solid tumors include those of breast, prostate, brain, lung, kidney, liver, stomach, intestine, colon, thyroid, thymus, ovary, melanoma, and pancreas among others. Replacement stem cells supply the function of endogenous HSPCs (e.g., in fighting infection) and if allogenic may have additional activity against residual cancer cells.

The present methods can also be used for replacing HSPCs in organ transplants, particularly allografts. Endogenous HSPCs are likely to develop a host verses graft response against non-MHC-matched allografts. The host versus graft response can be reduced by ablating endogenous HSPCs before the organ transplant and introducing replacement HSPCs genetically modified to confer a proliferation advantage at the same time as the transplanted organ and preferably from the same source (i.e., subject).

Selection between autologous and allogenic sources for replacement HSPCs depends on several factors. Autologous transplantation is readily available, and there is no need to identify an HLA-matched donor. Autologous transplants have a lower risk of life-threatening complications; there is no risk of GVHD and no need for immunosuppressive therapy to prevent GVHD and graft rejection. Immune reconstitution is more rapid than after an allogeneic transplant and there is a lower risk of opportunistic infections. Graft failure occurs rarely. However, there is a risk that autologous transplants from cancer patients are contaminated with cancer cells.

Allogeneic transplantation has the advantage that the graft is free of contaminating tumor cells. The graft also includes donor-derived immunocompetent cells which may produce an immune graft-versus-malignancy effect. There is generally a lower risk for disease recurrence after allogeneic transplants compared to autologous transplantation. However, allogeneic transplants may be associated with a number of potentially fatal complications such as regimen-related organ toxicity, graft failure, and graft-versus-host disease.

In general, allogeneic transplants have been used predominantly in the treatment of leukemias and myelodysplastic syndromes. Autologous transplants have been used more often in solid tumors, lymphoma, and myeloma. For correction of genetic disorders, autologous transplants can be bused with genetic modification to correct the genetic basis for the disorder or allogenic transplant without the need for correction.

### VIII. Regimes for Administering Replacement Stem Cells

Replacement stem cells are administered parenterally, typically by intravenous infusion. The dose of stem cells administered can depend on the desired purity of the infused cell composition, and the source of the cells. The dose can also depend on the type of genetic modification of the HSPCs. Because of the protection of HSPCs and because substantially complete elimination of endogenous HSPCs before introduction of replacement HSPCs is not necessary, dosages can sometimes be less than in prior methods in which 1-2×10⁶ CD34+ cells/kg body weight was considered a minimum. Exemplary dosages of cells for reintroduction are at least 1×10⁵, 1×10⁶, 2,×10⁶, 5×10⁶, 10⁷, 2×10⁷ CD34+ cells/kg body weight. Exemplary ranges are 1×10⁵ to 5×10⁷, 1×10⁶ to 2×10⁷, or 5×10⁵-6×10⁶ CD34+cells/kg body weight. The dose may be limited by the number of available cells. Typically, regardless of the source, the dose is calculated by the number of CD34⁺ cells present. The percent number of CD34⁺ cells can be low for unfractionated bone marrow or mobilized peripheral blood; in which case the total number of cells administered is much higher.

### IX. Monitoring

After introduction of genetically modified replacement HSPCs into a subject, the ratio of replacement HSPCs to total HSPCs can be monitored. A sample of HSPCs can be obtained from bone marrow or peripheral blood as previously described. Replacement HSPCs can be distinguished from endogenous by e.g., a nucleic acid hybridization assay or immunoassays. If the replacement HSPCs are allogenic or xenogenic, there are many genetic differences a between the replacement and endogenous cells that can form the basis of a differential probe binding assay and sometimes differences in receptors that allow an immunoassay. If the replacement HSPCs are autologous, the genetic modification of the replacement HSPCs can distinguish them from endogenous HSPCs by either a nucleic acid hybridization assay or immunoassay. The proportion of replacement to total HSPCs may increase with time after introduction. Preferably the proportion exceeds 30, 50, 75, 90 or 95% after six months.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

### EXAMPLES

### Example 1

### MATERIALS AND METHODS

### Animals

Rhesus macaque (macaca mulatta) were used in these studies (2-4 year-old females, 2-4 kg) were housed and handled and procedures were conducted in accordance to the guidelines outlined in a protocol approved by the animal care and use committee. Bone marrow aspirates were collected in an alternating manner from the grater trochanter or humerus on days -7, 2, 9, 16, 29 and day 57.

### Reagents

FACS buffer (PBS, 2% FBS (Life Technologies), 2 mM EDTA (Life Technologies). Ultra rainbow beads (Spherotech). Antibodies used: CD34 PE (clone 563), CD45RA APCH7 (clone 5H9), CD45 FITC (clone D058-1283), CD90 V605 (clone 5E10), CD3 PerCPCy5.5 (clone SP34), CD20 PerCPCy5.5 (clone 2H7), CD16 PerCPCy5.5 (clone 3G8), CD11b PerCPCy5.5 (clone ICRF44), CD4 PerCPCy5.5 (clone L200), CD8a (PerCPCy5.5 clone RPA-T8), purified anti-CD32 (clone FLI8.26). Pharm Lyse 10× prepared at 1× with purified water. Sytox Blue and purified water (Life Technologies). Unconjugated humanized SR1 (Forty Seven, Inc.), Unconjugated magrolimab (Forty Seven Inc.), Anti-IgG-AF647 (clone 4E3, Southern Biotech), Anti-IgG4-AF647 (clone G17-4, Forty Seven Inc.)

### Immunophenotyping of Bone Marrow Aspirates

Bone marrow was washed with FACS buffer and pelleted. Red blood cells were lysed with 1× Pharm lyse for 10-15 minutes at room temperature washed into FACS buffer. Cells are then blocked with anti-CD32 for 10-15 minutes on ice then washed into FACS buffer. Cells were immunophenotyped by staining the cells with CD34, CD45, CD45RA, CD90 and lineage (lin) markers: CD3, CD4, CD8a, CD11b, CD20, CD16 for 30 minutes on ice. Cells were then washed again with FACs buffer and a viability stain (Sytox Blue) was added before acquiring on the flow cytometer. Target cell populations of interest are defined as sytox(−)Lin(−)CD34(+)CD45(mid)CD90(+)CD45RA(−)cKIT(+), sytox(−)Lin(−)CD34(+)CD45(mid)CD90(+)CD45RA(−)CD47(+), sytox(−)Lin(−) CD34(+)CD45(mid)CD90(+)CD45RA(−), sytox(−)Lin(−)CD34(+)CD90(+), sytox(−)Lin(−)CD34(+)cKIT(+).

### Calculation of Target Cell Population Change

Target reference cell population was assessed for each animal at day -7 and is the target cell reference value (Target_{reference}). Target_{reference} is calculated by the target population cell number divided by the cell number of sytox(−), sytox(−)Lin(−)CD34(+), sytox(−)Lin(−), sytox(−)Lin(−)CD34(+), sytox(−)Lin(−)CD34(+)CD90(+) or sytox(−)Lin(−)CD34(+)cKIT(+). Target_{time point} is calculated for each time point (t=2, 9, 16, 29, and 57) in the same manner as carried out as Target reference. Each animal is normalized by calculating % change of target cell population from start=(Target_{time point}/Target_{reference})*100.

Receptor Occupancy for cKIT

Bone marrow aspirate red blood cells were lysed with 1× Pharm lyse for 10-15 minutes at room temperature washed with FACs buffer. Cells are then blocked with anti-CD32 for 10-15 minutes on ice then washed into FACs buffer. To measure total cKIT receptors, cells are incubated with unconjugated anti-c-kit (humanized SR1) (5 ug/ml) for 35 minutes on ice, washed twice with FACs buffer, stained with anti-IgG1-AF647 (50 ug/ml) for 20 minutes on ice, washed twice then cells are stained with the immunophenotyping cocktail protocol before acquisition on the flow cytometer. To measure occupied receptors prepared cells (lysed and blocked) are incubated without additional humanized SR1 saturation and stained with anti-IgG1-AF647 (50 ug/ml), incubated on ice for 20 minutes, washed twice then stained with the immunophenotyping cocktail protocol.

### Calculation of Receptor Occupancy

Receptor Occupancy will be calculated as follows:

RO Calculation: MFIₜₑₛₜ/MFIₜₒₜₐₗ multipled by 100%. MFIₜₒₜₐₗ is from the 5 ug/ml FSI-174 stained tube from same day of assessment as MFIₜₑₛₜ.

### Results

FIGS. 1A, B shows the basic treatment protocol. FIG. 1A shows PBS control and individual treatment with either anti-c-kit (humanized SR1) or anti-CD47 (magrolimab). FIG. 1B shows PBS control and combination treatment with anti-c-kit and anti-CD47. Different dosages of anti-C-kit ranging from 0.3 mg/kg to 3 mg/kg were tested as shown. The priming dose of anti-CD47 was 5 mg/kg and subsequent dose 20 mg/kg.

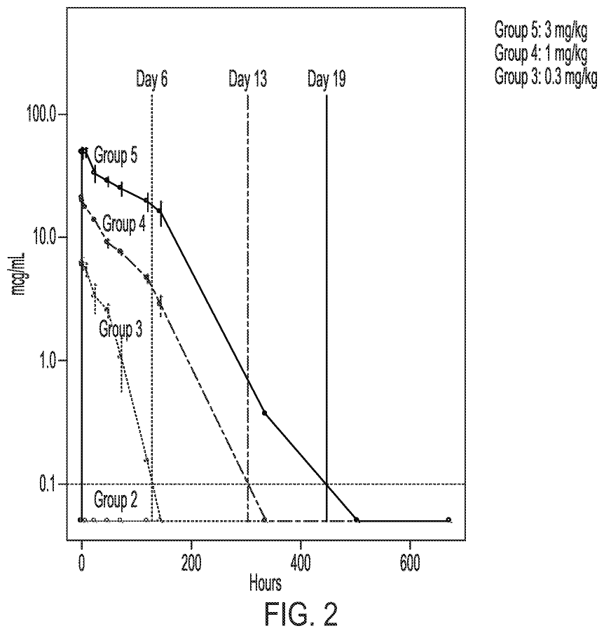

FIG. 2 shows serum concentration of anti-c-kit with time. All doses achieve measurable serum levels above the desired threshold of 0.1 μg/ml. Dose 0.3 mg/kg dropped after 6 days below minimal measurable serum level, dose 1 mg/kg after 13 days, and dose 3 mg/kg after 19 days.

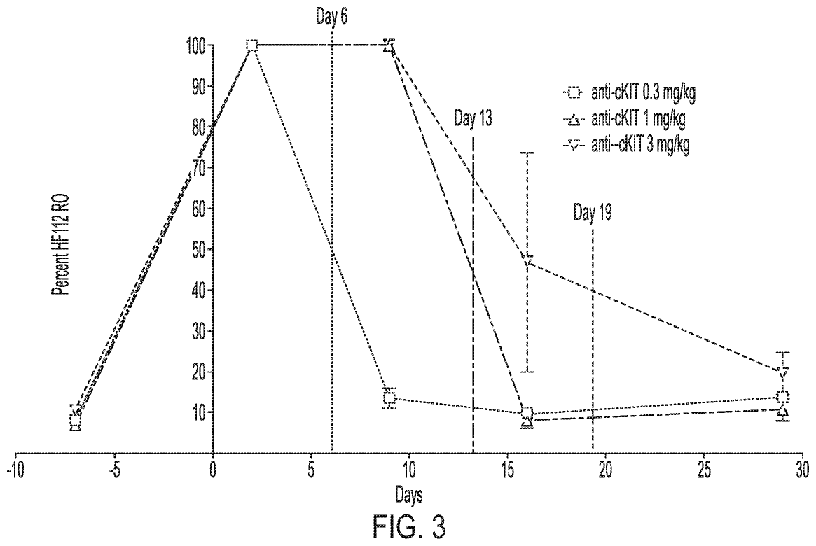

FIG. 3 shows c-kit receptor occupancy for different dosages of anti-c-kit. All dosages achieved 100% c-kit receptor occupancy of HSCs.

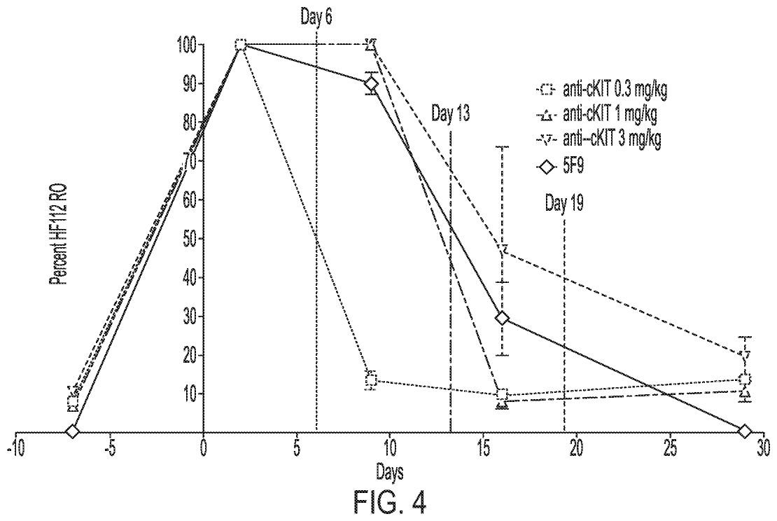

FIG. 4 shows anti-c-kit and anti-CD47 receptor occupancy with time. All dosages of anti-c-kit achieved 100% receptor occupancy on HSCs as did the 5 mg/kg-20 mg/kg doses of anti-CD47.

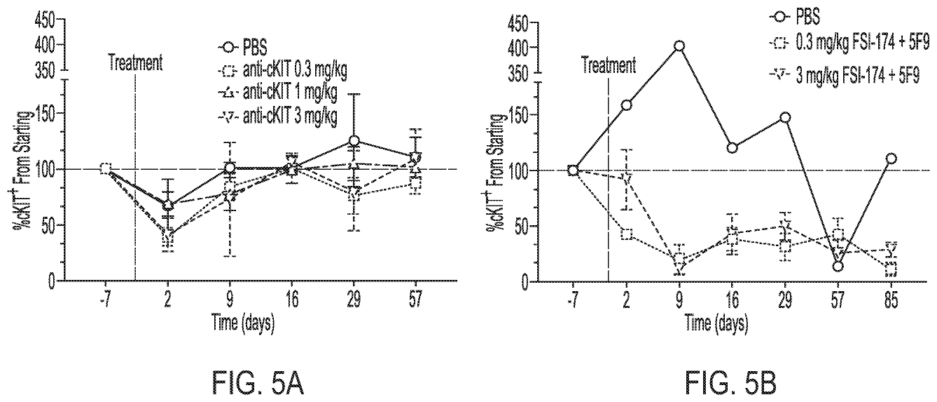

FIGS. 5A, B show % changes in c-kit positive HSCs relative to baseline. Treatment with anti-c-kit alone did not result in significant decrease relative to the negative control, whereas treatment with the combination of anti-c-kit and anti-CD47 resulted in a significant decrease. The decrease was not significantly different for 0.3 and 3 mg/kg anti-c-kit.

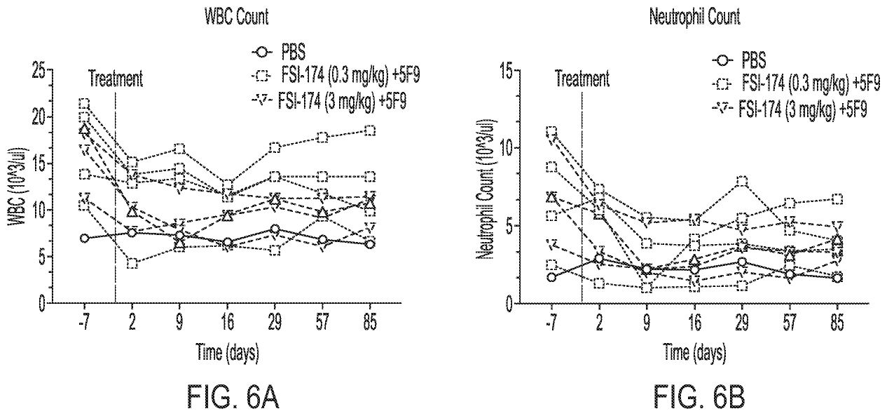

FIGS. 6A, B show changes in peripheral white blood cells or neutrophils over time with combination treatment with anti-c-kit and anti-CD47. Treatment did not result in significant decline of white blood cells or neutrophils relative to the negative control. No neutropenia or pancytopenias were observed.

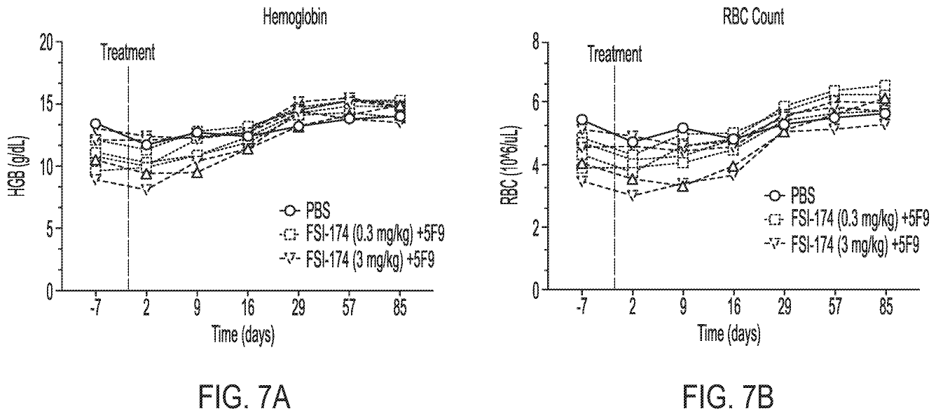

FIGS. 7A, B show hemoglobin and red blood cells levels over time for combined anti-c-kit anti-CD47 treatment relative to a negative control. Treatment results in a mild and transient anemia due to anti-CD47 elimination of aged red blood cells.

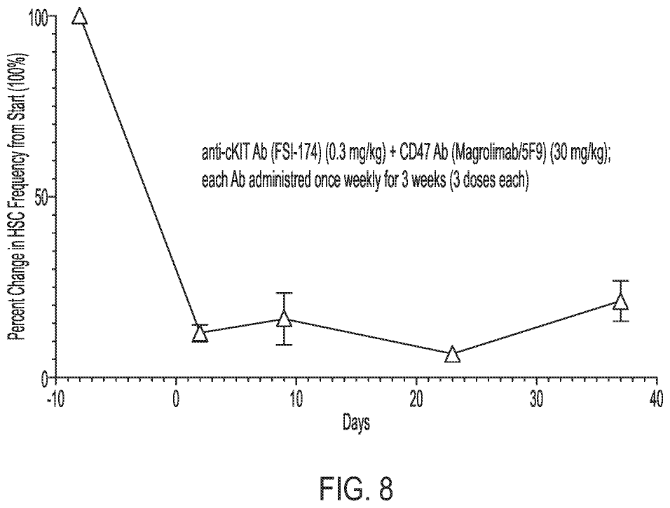

In a further experiment, rhesus monkey were dosed with anti-c-kit and anti-CD47 antibodies on day 1, day 8 and day 15. Bone marrow aspirates were harvested on day-6, 2, 9, 23, 37 and analyzed by flow cytometry for hematopoietic stem and progenitor cell frequency. All monkeys received a CD47 ab priming dose one week prior to the therapeutic dosing. FIG. 8 shows about 85-90% reductions in HSC's from days 2-25 with recovery thereafter.

### Example 2

This example shows HSPC cell depletion with a combination of antibodies against c-kit and SIRPα.

### Methods:

Transgenic mice expressing human SIRPα were injected with 400 ug of anti-SIRPα (1H9) intraperitoneally on day-6, -4 and day -2 or mice were injected with 500 ug of anti-c-kit (ACK2) intravenously on day-6. Bone marrow was harvested on day 0 and stained for hematopoietic stem and progenitor cells and assessed by flow cytometry.

HSC depletion was evaluated in bone marrow with staining panels as follows:

TABLE 2
FACS STAINING PANEL

| | | | Dilution | |
|---|---|---|---|---|
| | Vendor and | | from stock | Concentration |
| MARKER | Catalog # | COLOR | tube | during staining |
| CD34 | BD 553733 | FITC | 1/100 | 5 ug/mL |
| CD135 | BioLegend | BV421 | 1/100 | 2 ug/mL |
| | 135314 | | | |
| CD150 | BioLegend | BV785 | 1/100 | 2 ug/mL |
| | 115937 | | | |
| SCA-1 | BioLegend | APC | 1/100 | 2 ug/mL |
| | 122512 | | | |
| LIN | BioLegend | AF700 | 1/50 | Lot: B284780 |
| | 133313 | | | |
| 2B8 | BioLegend | PE | 1/100 | 2 ug/mL at |
| | 105808 | | | 1/100 |
| 7AAD | BD 559925 | PerCP-Cy5.5 | | 5 uL per well |
| Thy1.2 | BioLegend | BV605 | 1/100 | 2 ug/mL |
| | 140318 | | | |

TABLE 3
BONE MARROW IMMUNOPHENOTYPING:
STAINING PANEL

| | | | Dilution | |
|---|---|---|---|---|
| | Vendor and | | from stock | Concentration |
| MARKER | Catalog # | COLOR | tube | during staining |
| CD3 | BioLegend | FITC | 1/100 | 5 ug/mL |
| | 100204 | | | |
| CD11b | BioLegend | APC | 1/100 | 2 ug/mL |
| | 101212 | | | |
| CD45 | BioLegend | AF700 | 1/100 | 5 ug/mL |
| | 103128 | | | |
| CD19 | BioLegend | BV421 | 1/100 | 2 ug/mL |
| | 115538 | | | |
| Gr1 | BioLegend | BV605 | 1/100 | 2 ug/mL |
| | 108440 | | | |
| 7AAD | BD 559925 | PerCP-Cy5.5 | 5 uL per well | |

### Plating and Staining

Multichannel pipette were used to distribute at least 2 million cells per well in a final volume of 100 uL. For samples with insufficient amount of cells, all cells were plated. FC was blocked with 4 uL/well for 5-10 minutes at 4° C. Purified Rat Anti-Mouse CD16/CD32 (Mouse BD Fc Block™) (BD Biosciences cat no: 553141). A Master mix was prepared of the full mouse HSC staining panel and mouse BM immunostaining panel. Mixtures were spun down at 4° C., 1600RPM, 5 minutes to pellet. The Fc was removed, without additional washing 100 uL of staining solution master mix was added to its respective samples and incubate for 90 min, in the dark, at 4° C. 100 uL of FACS buffer was added followed by spinning down at 4 C, 1600 RPM, 5 minutes, on a floor top centrifuge. Two more washes were performed with 200 uL of FACS buffer. Resuspension was performed in 250uL of FACS buffer+5 uL of 7AAD per well.

### Results

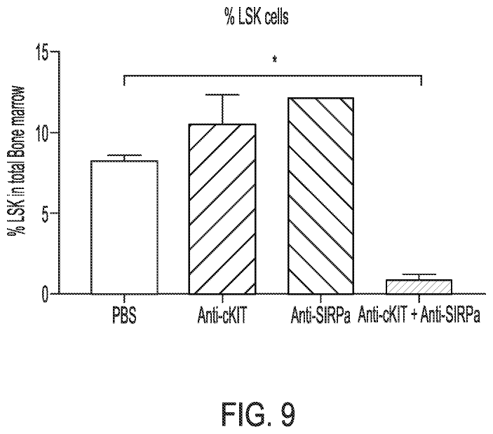

Mice expressing human SIRPα were injected with 400 ug of anti-SIRPα antibody (humanized 1H9) on days-6, -4 and day -2 or mice were injected with 500 ug anti-c-kit (humanized SR1) on day-6. This proof of concept experiment was designed to test anti-SIRPα in combination with anti-c-kit to deplete HSPCs at doses that would allow saturation of receptors. Bone marrow was harvested on day 0 and analyzed by flow cytometry for HSPC cells, which are Lineage negative, Sca-1 positive, c-kit positive cells (and can also be referred to as LKS cells). FIG. 9 shows that combination of anti-c-kit and anti-SIRPα is highly efficacious in depletion of HSPCs from the bone marrow.

1. A method of ablating hematopoietic stem and progenitor cells (HSPCs) in a patient in need thereof, comprising administering to the patient 0.15-2 mg/kg of an immunotherapeutic agent specifically binding to c-kit and an effective regime of an immunotherapeutic agent specifically binding to CD47 or SIRPα, wherein HSPCs are ablated in the patient.

2. The method of claim 1, wherein the patient is administered a single dose of 0.15-1 mg/kg of the immunotherapeutic agent specifically binding to c-kit.

3. The method of claim 1, wherein the patient is administered the immunotherapeutic agent specifically binding to c-kit in multiple doses over a period of up to seven days delivering substantially the same area under the curve as a single dose of 0.15-1 mg/kg.

4. The method of claim 1, wherein the patient is administered two doses of 0.15-1 mg/kg of the immunotherapeutic agent specifically binding to c-kit spaced by 3-7 days.

5. The method of claim 1, wherein that the immunotherapeutic agent specifically binding to CD47 is administered and the effective regime of an immunotherapeutic agent specifically binding to CD47 comprises a first dose and a second dose higher than first dose.

6. The method of claim 5, wherein the first dose is 1 mg/kg and the second dose is 10-30 mg/kg, preferably 15-20 mg/kg.

7. The method of claim 5, wherein the immunotherapeutic agent specifically binding to c-kit is administered as a single dose at the same time as the second dose of the immunotherapeutic agent specifically binding to CD47.

8. The method of claim 7, wherein the single dose of the immunotherapeutic agent specifically binding to c-kit and the second dose of the immunotherapeutic agent specifically binding to CD47 are administered by co-infusion.

9. The method of claim 7, wherein the second dose of immunotherapeutic specifically binding to CD47 and single dose of immunotherapeutic specifically binding to c-kit are administered 3-15 days, optionally 7 days after the first dose of the immunotherapeutic agent specifically binding to CD47.

10. The method of claim 1, wherein the immunotherapeutic agent specifically binding to c-kit is administered in at least three doses over a period of 10-30 days.

11. The method of claim 10, wherein the immunotherapeutic agent specifically binding to CD47 or SIRPα is administered on the same day as each dose of the immunotherapeutic agent specifically binding to c-kit is administered, optionally with an additional dose lower than and preceding the other dosages.

12. The method of claim 1, further comprising introducing HSPCs into the patient.

13. The method of claim 7, wherein HSPCs are introduced into the patient 5-15 days after the single dose of the immunotherapeutic agent specifically binding to c-kit and the second dose of the immunotherapeutic agent specifically binding to CD47 are administered.

14. The method of claim 13, wherein only the first and second doses of immunotherapeutic agent specifically binding to CD47 and the single dose of immunotherapeutic agent specifically binding to c-kit are administered before introducing the HSPCs.

15. The method of claim 5, further comprising administering a third dose of the immunotherapeutic agent specifically binding to CD47 after the second dose, optionally the second and third doses being the same amount of the immunotherapeutic agent.

16. The method of claim 1, wherein multiple doses of the immunotherapeutic agent specifically binding to c-kit are administered and multiple doses of the immunotherapeutic agent specifically binding to CD47 or SIRPα are administered and the HSPC's are introduced into the patient 5-15 days after the last dose of the immunotherapeutic agent specifically binding to c-kit, or the immunotherapeutic agent specifically binding to CD47 or SIRPα, whichever is later.

17. The method of claim 16, wherein the last dose of the immunotherapeutic agent specifically binding to c-kit and the last dose of the immunotherapeutic agent specifically binding to SIRPα are administered on the same day.

18. The method of claim 1, wherein the immunotherapeutic agent specifically binding to CD47 is an antibody specifically binding to CD47.

19. The method of claim 18, wherein the immunotherapeutic agent specifically binding to CD47 is humanized 5F9.

20. The method of claim 19, wherein the antibody is magrolimab.

21-27. (canceled)

28. The method of claim 1, wherein the immunotherapeutic agent specifically binding to c-kit is an antibody.

29. The method of claim 28, wherein the antibody is a humanized form of SR1 of human IgG1 isotype.

30. The method of claim 29, wherein the antibody comprises a heavy chain variable region having a sequence comprising any of SEQ ID NOS:7-9 and a light chain variable region having a sequence comprising SEQ ID NO:10.

31. The method of claim 30, wherein the heavy chain variable region has a sequence comprising SEQ ID NO:7.

32. The method of claim 1, wherein administration of the immunotherapeutic agents ablates c-kit positive HSPCs by 25-95% of the level before administration.

33. The method of claim 1, wherein administration of the immunotherapeutic agents ablates c-kit positive HSPCs by 25-75% of the level before administration.

34. The method of claim 1, wherein the patient has a hematologic cancer treated by ablation of the HSPC's.

35. The method of claim 34, wherein the patient is also administered an agent effective to treat the hematologic cancer.

36. The method of claim 34, wherein the patient is administered the agent the before or during ablation of the HSPCs.

37. The method of claim 34, wherein the agent is a chemotherapeutic agent, anti-angiogenic agent, anti-fibrotic agent or monoclonal antibody against a cancer antigen.

38. The method of claim 34, wherein the hematologic cancer is a lymphoma, leukemia or myeloma.

39. The method of claim 1, wherein the patient has a solid tumor and the patient is administered an agent effective to treat the solid tumor, and which damages HSPCs of the patient, before ablating the HSPCs in the patient.

40. The method of claim 39, wherein the agent is a chemotherapeutic agent.

41. The method of claims 1, wherein a CAR-T cell is administered to the patient after ablating the HSPCs.

42. The method of claim 1, further comprising administering a flt3 agonist or CISH inhibitor after ablation of the HSPCs to promote growth of HSPCs or a cellular therapy.

43. The method of claim 1, further co-administering an MCL1 inhibitor with the immunotherapeutic agent specifically binding to c-kit and immunotherapeutic agent specifically binding to CD47 or SIRPα to ablate NK cells.

44. The method of claim 1, wherein the patient is a human.

45-47 (canceled)


SEQUENCE LISTING (58 sequences)

SEQ ID NO: 1 - protein, 5 aa, Artificial sequence
  Synthesized
SYNMH

SEQ ID NO: 2 - protein, 17 aa, Artificial sequence
  Synthesized
VIYSGNGDTSYNQKFKG

SEQ ID NO: 3 - protein, 8 aa, Artificial sequence
  Synthesized
ERDTRFGN

SEQ ID NO: 4 - protein, 15 aa, Artificial sequence
  Synthesized
RASESVDIYGNSFMH

SEQ ID NO: 5 - protein, 7 aa, Artificial sequence
  Synthesized
LASNLES

SEQ ID NO: 6 - protein, 9 aa, Artificial sequence
  Synthesized
QQNNEDPYT

SEQ ID NO: 7 - protein, 117 aa, Artificial sequence
  Synthesized
QVQLVQSGAEVKKPGASVKVSCKASGYTFTSYNMHWVRQAPGQGLEWMGVIYSGNGDTSYAQKFKGRVTITADKSTSTAYMELSSLRSEDTAVYYCARERDTRFGNWGQGTLVTVSS

SEQ ID NO: 8 - protein, 117 aa, Artificial sequence
  Synthesized
QVQLVQSGAEVKKPGASVKVSCKASGYTFTSYNMHWVRQAPGQGLEWMGVIYSGNGDTSYNQKFQGRVTITADKSTSTAYMELSSLRSEDTAVYYCARERDTRFGNWGQGTLVTVSS

SEQ ID NO: 9 - protein, 117 aa, Artificial sequence
  Synthesized
QVQLVQSGAEVKKPGASVKVSCKASGYTFTSYNMHWVRQAPGQGLEWMGVIYSGNGDTSYAQKFQGRVTITADKSTSTAYMELSSLRSEDTAVYYCARERDTRFGNWGQGTLVTVSS

SEQ ID NO: 10 - protein, 111 aa, Artificial sequence
  Synthesized
DIVMTQSPLSLPVTPGEPASISCRASESVDIYGQSFMHWYLQKPGQSPQLLIYLASNLESGVPDRFSGSGSGTDFTLKISRVEAEDVGVYYCQQNNEDPYTFGGGTKVEIK

SEQ ID NO: 11 - protein, 5 aa, Artificial Sequence
  5F9 heavy chain CDR1
NYNMH

SEQ ID NO: 12 - protein, 17 aa, Artificial Sequence
  5F9 heavy chain CDR2
TIYPGNDDTSYNQKFKD

SEQ ID NO: 13 - protein, 8 aa, Artificial Sequence
  5F9 heavy chain CDR3
GGYRAMDY

SEQ ID NO: 14 - protein, 16 aa, Artificial Sequence
  5F9 light chain CDR1
RSSQSIVYSNGNTYLG

SEQ ID NO: 15 - protein, 7 aa, Artificial Sequence
  5F9 light chain CDR2
KVSNRFS

SEQ ID NO: 16 - protein, 9 aa, Artificial Sequence
  5F9 light chain CDR3
FQGSHVPYT

SEQ ID NO: 17 - protein, 117 aa, Artificial Sequence
  Humanized antibody hu5F9-vh1
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 18 - protein, 117 aa, Artificial Sequence
  Humanized antibody hu5F9-vh2
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 19 - protein, 117 aa, Artificial Sequence
  Humanized antibody hu5F9-vh3
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 20 - protein, 112 aa, Artificial Sequence
  Humanized antibody hu5F9-vl1
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 21 - protein, 112 aa, Artificial Sequence
  Humanized antibody hu5F9-vl2
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 22 - protein, 112 aa, Artificial Sequence
  Humanized antibody hu5F9-vl3
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK

SEQ ID NO: 23 - protein, 5 aa, Artificial sequence
  1H9 CDR-H1
SYWIT

SEQ ID NO: 24 - protein, 17 aa, Artificial sequence
  1H9 CDR-H2
DIYPGSGSTNHIEKFKS

SEQ ID NO: 25 - protein, 11 aa, Artificial sequence
  1H9 CDR-H3
GYGSSYGYFDY

SEQ ID NO: 26 - protein, 11 aa, Artificial sequence
  1H9 CDR-L1
RASENIYSYLA

SEQ ID NO: 27 - protein, 7 aa, Artificial sequence
  1H9 CDR-L2
TAKTLAE

SEQ ID NO: 28 - protein, 9 aa, Artificial sequence
  1H9 CDR-L3
QHQYGPPFT

SEQ ID NO: 29 - protein, 120 aa, Artificial sequence
  Humanized 1H9 VH
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 30 - protein, 107 aa, Artificial sequence
  Humanized 1H9 VL
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 31 - protein, 5 aa, Artificial sequence
  3C2 CDR-H1
SYWMH

SEQ ID NO: 32 - protein, 17 aa, Artificial sequence
  3C2 CDR-H2
NIDPSDSDTHYNQKFKD

SEQ ID NO: 33 - protein, 10 aa, Artificial sequence
  3C2 CDR-H3
GYSKYYAMDY

SEQ ID NO: 34 - protein, 16 aa, Artificial sequence
  3C2 CDR-L1
RSSQSIVHSYGNTYLE

SEQ ID NO: 35 - protein, 7 aa, Artificial sequence
  3C2 CDR-L2
KVSNRFS

SEQ ID NO: 36 - protein, 9 aa, Artificial sequence
  3C2 CDR-L3
FQGSHVPYT

SEQ ID NO: 37 - protein, 119 aa, Artificial sequence
  Humanized 3C2 VH
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 38 - protein, 112 aa, Artificial sequence
  Humanized 3C2 VL
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 39 - protein, 449 aa, Artificial sequence
  Humanized 1H9 HC (full-length)
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 214 aa, Artificial sequence
  Humanized 1H9 LC (full-length)
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 448 aa, Artificial sequence
  Humanized 3C2 HC (full-length)
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 219 aa, Artificial sequence
  Humanized 3C2 LC (full-length)
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 5 aa, Artificial sequence
  9B11 CDR-H1
DYYIH

SEQ ID NO: 44 - protein, 17 aa, Artificial sequence
  9B11 CDR-H2
RIDPEDGETKYAPKFQG

SEQ ID NO: 45 - protein, 5 aa, Artificial sequence
  9B11 CDR-H3
GGFAY

SEQ ID NO: 46 - protein, 11 aa, Artificial sequence
  9B11 CDR-L1
ASSSVSSSYLY

SEQ ID NO: 47 - protein, 7 aa, Artificial sequence
  9B11 CDR-L2
STSNLAS

SEQ ID NO: 48 - protein, 9 aa, Artificial sequence
  9B11 CDR-L3
HQWSSHPYT

SEQ ID NO: 49 - protein, 114 aa, Artificial sequence
  9B11 VH
EVQLQQSGAELVKPGASVKLSCTASGFNIKDYYIHWVKQRTEQGLEWIGRIDPEDGETKYAPKFQGKATITADTSSNTAYLQLNSLTSEDTAVYSCAKGGFAYWGQGTLVTVSA

SEQ ID NO: 50 - protein, 108 aa, Artificial sequence
  9B11 VL
QIVLTQSPAIMSASPGEKVTLTCSASSSVSSSYLYWYQQKPGSSPKLWIYSTSNLASGVPARFSGSGSGTSYSLTISSMEAEDAASYFCHQWSSHPYTFGGGTKLEIK

SEQ ID NO: 51 - protein, 5 aa, Artificial sequence
  7E11 CDR-H1
SYWMH

SEQ ID NO: 52 - protein, 17 aa, Artificial sequence
  7E11 CDR-H2
NIDPSDSDTHYNQKFKD

SEQ ID NO: 53 - protein, 12 aa, Artificial sequence
  7E11 CDR-H3
SYGNYGENAMDY

SEQ ID NO: 54 - protein, 16 aa, Artificial sequence
  7E11 CDR-L1
RSSQSIVHSYGNTYLE

SEQ ID NO: 55 - protein, 7 aa, Artificial sequence
  7E11 CDR-L2
KVSNRFS

SEQ ID NO: 56 - protein, 9 aa, Artificial sequence
  7E11 CDR-L3
FQGSHVPFT

SEQ ID NO: 57 - protein, 120 aa, Artificial sequence
  7E11 VH
QVKLQESGAELVRPGSSVKLSCKASGYTFTSYWMHWVKQRPIQGLEWIGNIDPSDSDTHYNQKFKDKATLTVDNSSSTAYMQLSSLTSEDSAVYYCASYGNYGENAMDYWGQGTSVTVSS

SEQ ID NO: 58 - protein, 112 aa, Artificial sequence
  7E11 VL
DILMTQTPLSLPVSLGDQASISCRSSQSIVHSYGNTYLEWYLQKPGQSPKLLIYKVSNRFSGVPDRFSGSGSGTDFTLKISRVEAEDLGVYYCFQGSHVPFTFGSGTKLEIK